(19) United States
(12) Patent Application Publication
(10) Pub. No.: US 2024/0374166 A1
(43) Pub. Date: Nov. 14, 2024

(54) INFORMATION PROCESSING DEVICE, ELECTRONIC DEVICE, INFORMATION PROCESSING SYSTEM, INFORMATION PROCESSING METHOD, AND PROGRAM

(71) Applicant: KYOCERA Corporation, Kyoto (JP)

(72) Inventors: Martin KLINKIGT, Shibuya-ku, Tokyo (JP); Hideyuki KIMPARA, Yokohama-shi, Kanagawa (JP); Naoki NISHIDA, Yokohama-shi, Kanagawa (JP); Takashi NAGATOMO, Kita-ku, Tokyo (JP); Edwardo Arata YAMAMOTO MURAKAMI, Kawasaki-shi, Kanagawa (JP); Takashi SUZUKI, Sagamihara-shi, Kanagawa (JP); Masayuki KISHI, Hiratsuka-shi, Kanagawa (JP)

(73) Assignee: KYOCERA Corporation, Kyoto (JP)

(21) Appl. No.: 18/690,641

(22) PCT Filed: Sep. 14, 2022
(86) PCT No.: PCT/JP2022/034480

§ 371 Date: Mar. 8, 2024

(30) Foreign Application Priority Data
Sep. 14, 2021 (JP) 2021-149733

(51) Int. Cl. A61B 5/11; A43B 3/44; A61B 5/00; H04R 1/10

(52) U.S. Cl. CPC A61B 5/112; A43B 3/44; A61B 5/002; A61B 5/6807; A61B 5/7267; A61B 2562/0252; H04R 1/1041

(57) ABSTRACT

An information processing device includes a controller. The controller is configured to acquire an estimated value of a ground reaction force applied to a user by using sensor data representing movement of a body part of the user and a trained model. The trained model is trained and outputs the estimated value of the ground reaction force when input with the sensor data.

### CROSS-REFERENCE TO RELATED APPLICATIONS

This application claims priority from Japanese Patent Application No. 2021-149733 filed in Japan on Sep. 14, 2021, and the entire disclosure of this application is hereby incorporated by reference.

### TECHNICAL FIELD

The present disclosure relates to an information processing device, an electronic device, an information processing system, an information processing method, and a program.

### BACKGROUND OF INVENTION

Heretofore, a known technology is for measuring a ground reaction force. For example, Patent Literature 1 discloses a ground reaction force meter that measures ground reaction force data representing changes in ground reaction force during walking motion. For example, Patent Literature 2 discloses a mobile ground reaction force measurement device that is worn on the foot of a subject.

### CITATION LIST

### Patent Literature

Patent Literature 1: Japanese Unexamined Patent Application Publication No. 2013-138783
Patent Literature 2: Japanese Unexamined Patent Application Publication No. 2010-127921

### SUMMARY

In an embodiment of the present disclosure, an information processing device includes a controller.

The controller is configured to acquire an estimated value of a ground reaction force applied to a user by using sensor data representing movement of a body part of the user and a trained model.

The trained model is trained and outputs the estimated value of the ground reaction force when input with the sensor data.

In an embodiment of the present disclosure, an electronic device includes a notification unit.

The notification unit is configured to report the estimated value of the ground reaction force acquired by the information processing device described above.

In an embodiment of the present disclosure, an information processing system includes an information processing device.

The information processing device is configured to acquire an estimated value of a ground reaction force applied to a user by using sensor data representing movement of a body part of the user and a trained model.

The trained model is trained and outputs the estimated value of the ground reaction force when input with the sensor data.

In an embodiment of the present disclosure, an information processing method includes acquiring an estimated value of a ground reaction force applied to a user by using sensor data representing movement of a body part of the user and a trained model. The trained model is trained and outputs the estimated value of the ground reaction force when input with the sensor data.

In an embodiment of the present disclosure, a program is configured to cause a computer to execute acquiring an estimated value of a ground reaction force applied to a user by using sensor data representing movement of a body part of the user and a trained model. The trained model is trained and outputs the estimated value of the ground reaction force when input with the sensor data.

### BRIEF DESCRIPTION OF THE DRAWINGS

FIG. 1 is a diagram illustrating the schematic configuration of an information processing system according to an embodiment of the present disclosure.

FIG. 2 is a diagram for describing a local coordinate system and a global coordinate system.

FIG. 3 is a diagram for describing a user's gait.

FIG. 4 is a functional block diagram illustrating the configuration of the information processing system illustrated in FIG. 1.

FIG. 5 is a graph illustrating estimated values of normalized ground reaction force.

FIG. 6 is a functional block diagram illustrating the configuration of a transformer.

FIG. 7 is a functional block diagram illustrating the configuration of “Multi-Head Attention”.

FIG. 8 is a functional block diagram illustrating the configuration of “Scaled Dot-Product Attention”.

FIG. 9 is a diagram illustrating an example of a combination of sensor data.

FIG. 10 is a graph of evaluation results.

FIG. 11 is a diagram illustrating subjects.

FIG. 12 is a graph of an example of measured values and estimated values of the ground reaction force of a subject.

FIG. 13 is a graph of an example of measured values and estimated values of the ground reaction force of a subject.

FIG. 14 is a graph of an example of measured values and estimated values of the ground reaction force of a subject.

FIG. 15 is a graph of an example of measured values and estimated values of the ground reaction force of a subject.

FIG. 16 is a graph of an example of measured values and estimated values of the ground reaction force of a subject.

FIG. 17 is a graph of an example of measured values and estimated values of the ground reaction force of a subject.

FIG. 18 is a graph of an example of measured values and estimated values of the ground reaction force of a subject.

FIG. 19 is a graph of an example of measured values and estimated values of the ground reaction force of a subject.

FIG. 20 is a graph of an example of measured values and estimated values of the ground reaction force of a subject evaluated as having a large center of gravity shift.

FIG. 21 is a graph of an example of measured values and estimated values of the ground reaction force of a subject evaluated as having a large center of gravity shift.

FIG. 22 is a graph of an example of measured values and estimated values of the ground reaction force of a subject evaluated as having a large center of gravity shift.

FIG. 23 is a graph of an example of measured values and estimated values of the ground reaction force of a subject evaluated as having a large center of gravity shift.

FIG. 24 is a graph of an example of measured values and estimated values of the ground reaction force of a subject evaluated as having a small center of gravity shift.

FIG. 25 is a graph of an example of measured values and estimated values of the ground reaction force of a subject evaluated as having a small center of gravity shift.

FIG. 26 is a graph of an example of measured values and estimated values of the ground reaction force of a subject evaluated as having a small center of gravity shift.

FIG. 27 is a graph of an example of measured values and estimated values of the ground reaction force of a subject evaluated as having a small center of gravity shift.

FIG. 28 is a graph of an example of estimated values of ground reaction force when different sensor data are used.

FIG. 29 is a graph illustrating the differences between the measured values and the estimated values of the ground reaction force illustrated in FIG. 28.

FIG. 30 is a graph of an example of estimated values of ground reaction force when different sensor data are used.

FIG. 31 is a graph illustrating the differences between the measured values and the estimated values of the ground reaction force illustrated in FIG. 30.

FIG. 32 is a graph of an example of estimated values of ground reaction force when different sensor data are used.

FIG. 33 is a graph illustrating the differences between the measured values and the estimated values of the ground reaction force illustrated in FIG. 32.

FIG. 34 is a graph of an example of estimated values of ground reaction force when different sensor data are used.

FIG. 35 is a graph illustrating the differences between the measured values and the estimated values of the ground reaction force illustrated in FIG. 34.

FIG. 36 is a graph of an example of estimated values of ground reaction force when different sensor data are used.

FIG. 37 is a graph illustrating the differences between the measured values and the estimated values of the ground reaction force illustrated in FIG. 36.

FIG. 38 is a graph of an example of estimated values of ground reaction force when different sensor data are used.

FIG. 39 is a graph illustrating the differences between the measured values and the estimated values of the ground reaction force illustrated in FIG. 38.

FIG. 40 is a graph of an example of estimated values of ground reaction force when different sensor data are used.

FIG. 41 is a graph illustrating the differences between the measured values and the estimated values of the ground reaction force illustrated in FIG. 40.

FIG. 42 is a graph of an example of estimated values of ground reaction force when different sensor data are used.

FIG. 43 is a graph illustrating the differences between the measured values and the estimated values of the ground reaction force illustrated in FIG. 42.

FIG. 44 is a flowchart illustrating operation of ground reaction force estimation processing performed by an electronic device illustrated in FIG. 1.

FIG. 45 is a functional block diagram illustrating the configuration of an information processing system according to another embodiment of the present disclosure.

FIG. 46 is a sequence diagram illustrating operation of estimation processing performed by the information processing system illustrated in FIG. 45.

### DESCRIPTION OF EMBODIMENTS

There is room for improvement in existing technologies for measuring ground reaction force. The present disclosure can provide an improved technology for measuring ground reaction forces.

Embodiments of the present disclosure are described below while referring to the drawings. In the components illustrated in the drawings below, the same symbols are used for the same components.

### (Configuration of System)

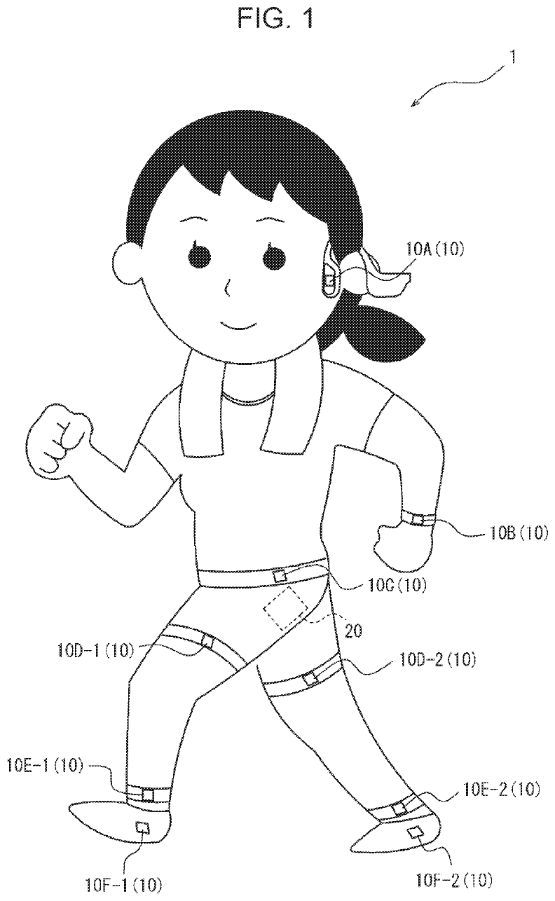

An information processing system 1 as illustrated in FIG. 1 can estimate the ground reaction force acting on a user while walking. The user may be walking in any location. Hereafter, the surface on which the user walks will be referred to as a “walking surface”. The walking surface is, for example, the ground, a road surface, or a floor surface.

The information processing system 1 includes a sensor device 10A, a sensor device 10B, a sensor device 10C, sensor devices 10D-1 and 10D-2, sensor devices 10E-1 and 10E-2, sensor devices 10F-1 and 10F-2, and an electronic device 20. However, the information processing system 1 does not need to include all of the sensor devices 10A, 10B, 10C, 10D-1, 10D-2, 10E-1, 10E-2, 10F-1, and 10F-1. The information processing system 1 only needs to include at least one from among the sensor devices 10A, 10B, 10C, 10D-1, 10D-2, 10E-1, 10E-2, 10F-1, and 10F-1.

Hereafter, when the sensor devices 10D-1 and 10D-2 are not particularly distinguished from each other, the sensor devices 10D-1 and 10D-2 will be collectively referred to as the “sensor device 10D”. When the sensor devices 10E-1 and 10E-2 are not particularly distinguished from each other, the sensor devices 10E-1 and 10E-2 will be collectively referred to as the “sensor device 10E”. When the sensor devices 10F-1 and 10F-2 are not particularly distinguished from each other, the sensor devices 10F-1 and 10F-2 will be collectively referred to as the “sensor device 10F”. When the sensor devices 10A to 10D are not particularly distinguished from each other, the sensor devices 10A to 10D will also be collectively referred to as the “sensor device 10”.

The sensor device 10 and the electronic device 20 can communicate with each other via communication lines. The communication lines include at least one out of wired and wireless communication lines.

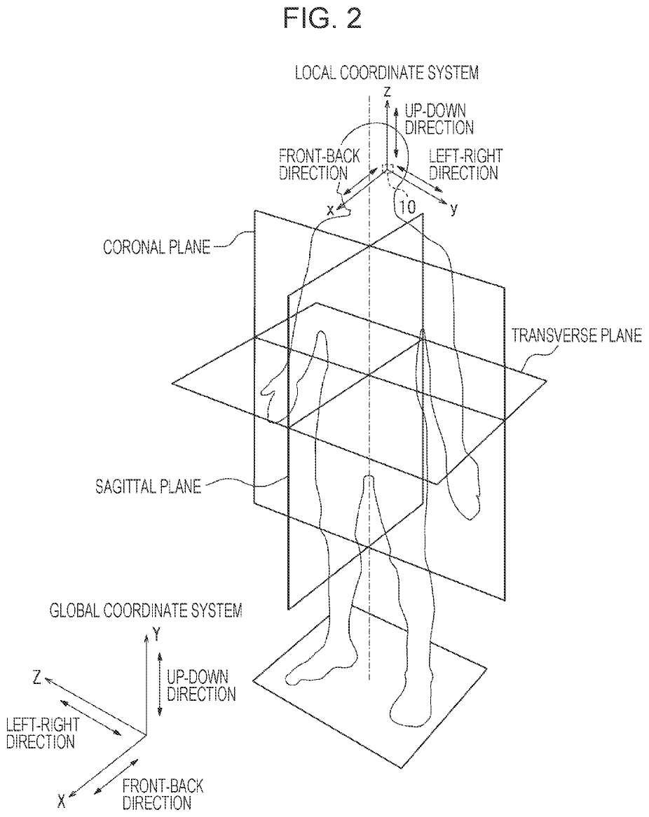

A local coordinate system is a coordinate system based on the positions of the sensor devices 10, as illustrated in FIG. 2. The position of the sensor device 10A is indicated in FIG. 2 with a dashed line as an example of the position of the sensor device 10. The local coordinate system consists of an x-axis, a y-axis, and a z-axis, for example. The x-axis, the y-axis, and the z-axis are orthogonal to one another. The x-axis is parallel to a front-back direction as viewed from the sensor device 10. The y-axis is parallel to a left-right direction as viewed from the sensor device 10. The z-axis is parallel to an up-down as viewed from the sensor device 10. The positive and negative directions of the x-axis, y-axis, and the z-axis may be set in accordance with the configuration of the information processing system 1 and so forth.

A global coordinate system is a coordinate system based on the position of the user in the space in which the user walks, as illustrated in FIG. 2. The global coordinate system consists of an X-axis, a Y-axis, and a Z-axis, for example. The X-axis, the Y-axis, and the Z-axis are orthogonal to one another. The X-axis is parallel to a front-back direction as seen from the user's perspective. The Y-axis is parallel to an up-down direction as seen from the user's perspective. The Z-axis is a parallel to a left-right direction as seen from the user's perspective. The positive and negative directions of the X-axis, Y-axis, and the Z-axis may be set in accordance with the configuration of the information processing system 1 and so on.

A sagittal plane is a plane that symmetrically divides the body of the user into a left section and a right section or a plane parallel to the plane that symmetrically divides the body of the user into a left section and a right section, as illustrated in FIG. 2. A coronal plane is a plane that divides the user's body into a front section and a rear section or a plane parallel to the plane that divides the user's body into a front section and a rear section. A transverse plane is a plane that divides the user's body into an upper section and a lower section or a plane that is parallel to the plane that divides the user's body into an upper section and a lower section. The sagittal plane, the coronal plane, and the transverse plane are perpendicular to each other.

As illustrated in FIG. 1, the sensor device 10 is worn on a body part of the user. The sensor device 10 detects sensor data indicating the movement of the body part on which the sensor device 10 is worn. The sensor data is data of the local coordinate system.

The sensor device 10A is worn on the user's head. For example, the sensor device 10A is worn on the user's ear. The sensor device 10A may be a wearable device. The sensor device 10A may be an earphone or may be contained in an earphone. Alternatively, the sensor device 10A may be a device that can be retrofitted to existing spectacles, earphones, or the like. The sensor device 10A may be worn on the user's head using any method. The sensor device 10A may be worn on the user's head by being incorporated into a hair accessory, such as a hair band or a hairpin, or an earring, a helmet, a hat, a hearing aid, a denture, or an implant.

The sensor device 10A may be worn on the user's head such that the x-axis of the local coordinate system based on the position of the sensor device 10A is parallel to the front-back direction of the head as seen from the user's perspective, the y-axis of the local coordinate system is parallel to the left-right direction of the head as seen from the user's perspective, and the z-axis of the local coordinate system is parallel to the up-down direction of the user's head as seen from the user's perspective. However, the x-axis, the y-axis, and the z-axis of the local coordinate system based on the position of the sensor device 10A do not necessarily need to be respectively aligned with the front-back direction, the left-right direction, and the up-down direction of the head as seen from the user's perspective. In this case, the relative orientation of the sensor device 10A with respect to the user's head may be initialized or identified as appropriate. The relative orientation may be initialized or identified by using information on the shape of a fixture used to attach the sensor device 10A to the user's head or by using captured image information of the user's head while the sensor device 10A is worn.

The sensor device 10A detects sensor data representing the movement of the user's head. The sensor data detected by the sensor device 10A includes, for example, at least any of the following: the velocity of the user's head, the acceleration of the user's head, the angle of the user's head, the angular velocity of the user's head, the temperature of the user's head, and the geomagnetism at the position of the user's head.

The sensor device 10B is worn on the user's forearm. For example, the sensor device 10B is worn on the user's wrist. The sensor device 10B may be worn on the user's left forearm or may be worn on the user's right forearm. The sensor device 10B may be a wristwatch-type wearable device. The sensor device 10B may be worn on the user's forearm by using any method. The sensor device 10B may be worn on the user's forearm by being incorporated into a band, a bracelet, a friendship bracelet, a glove, a ring, an artificial fingernail, a prosthetic hand, and so on. The bracelet may be a bracelet worn by the user as a decorative item or may be a bracelet that allows the user to wear a key such as a locker key on his or her wrist.

The sensor device 10B may be worn on the user's forearm such that the x-axis of the local coordinate system based on the position of the sensor device 10B is parallel to the front-back direction of the wrist as seen from the user's perspective, the y-axis of the local coordinate system is parallel to the left-right direction of the wrist as seen from the user's perspective, and the z-axis of the local coordinate system is parallel to the rotational direction of the wrist as seen from the user's perspective. The rotational direction of the wrist is, for example, the direction in which the wrist twists and turns.

The sensor device 10B detects sensor data representing the movement of the user's forearm. For example, the sensor device 10B detects sensor data representing movement of the wrist. The sensor data detected by the sensor device 10B includes, for example, at least any of the following: the velocity of the user's forearm, the acceleration of the user's forearm, the angle of the user's forearm, the angular velocity of the user's forearm, the temperature of the user's forearm, and the geomagnetism at the position of the user's forearm.

The sensor device 10C is worn on the user's waist. The sensor device 10C may be a wearable device. The sensor device 10C may be worn on the user's waist by using a belt, a clip, or the like.

The sensor device 10C may be worn on the user's waist such that the x-axis of the local coordinate system based on the position of the sensor device 10C is aligned with the front-back direction of the waist as seen from the user's perspective, the y-axis of the local coordinate system is aligned with the left-right direction of the waist as seen from the user's perspective, and the z-axis of the local coordinate system is aligned with the rotational direction of the waist as seen from the user's perspective. The rotational direction of the waist is, for example, the direction in which the waist twists and turns.

The sensor device 10C detects sensor data representing the movement of the user's waist. The sensor data detected by the sensor device 10C includes, for example, at least any of the following: the velocity of the user's waist, the acceleration of the user's waist, the angle of the user's waist, the angular velocity of the user's waist, the temperature of the user's waist, and the geomagnetism at the position of the user's waist.

The sensor device 10D-1 is worn on the user's left thigh. The sensor device 10D-2 is worn on the user's right thigh. The sensor device 10D may be a wearable device. The sensor device 10D may be worn on the user's thigh by using any method. The sensor device 10D may be worn on the user's thigh using a belt, a clip, or the like. The sensor device 10D may be worn on the thigh by being placed in a pocket, which is in the vicinity of the thigh, of the pants worn by the user. The sensor device 10D may be worn on the user's thigh by being incorporated into pants, underwear, shorts, a supporter, a prosthetic leg, an implant, and so on.

The sensor device 10D may be worn on the user's thigh such that the x-axis of the local coordinate system based on the position of the sensor device 10D is parallel to the front-back direction of the thigh as seen from the user's perspective, the y-axis of the local coordinate system is parallel to the left-right direction of the thigh as seen from the user's perspective, and the z-axis of the local coordinate system is parallel to the rotational direction of the thigh as seen from the user's perspective. The rotational direction of the thigh is, for example, the direction in which the thigh twists and turns.

The sensor device 10D-1 detects sensor data representing the movement of the user's left thigh. The sensor device 10D-2 detects sensor data representing the movement of the user's right thigh. The sensor data detected by the sensor device 10D includes, for example, at least any of the following: the velocity of the user's thigh, the acceleration of the user's thigh, the angle of the user's thigh, the angular velocity of the user's thigh, the temperature of the user's thigh, and the geomagnetism at the position of the user's thigh.

The sensor device 10E-1 is worn on the user's left ankle. The sensor device 10E-2 is worn on the user's right ankle. The sensor device 10E may be a wearable device. The sensor device 10E may be worn on the user's ankle using any method. The sensor device 10E may be worn on the user's ankle using a belt, a clip, or the like. The sensor device 10E may be worn on the user's ankle by being incorporated into an anklet, a band, a friendship bracelet, a tattoo sticker, a supporter, a cast, a sock, a prosthetic leg, an implant, and so on.

The sensor device 10E may be worn on the user's ankle so that the x-axis of the local coordinate system based on the position of the sensor device 10E is aligned with the front-back direction of the ankle as seen from the user's perspective, the y-axis of the local coordinate system is aligned with the left-right direction of the ankle as seen from the user's perspective, and the z-axis of the local coordinate system is aligned with the rotational direction of the ankle as seen from the user's perspective. The rotational direction of the ankle is, for example, the direction in which the ankle twists and turns.

The sensor device 10E-1 detects sensor data representing the movement of the user's left ankle. The sensor device 10E-2 detects sensor data representing the movement of the user's right ankle. The sensor data detected by the sensor device 10E includes, for example, at least any of the following: the velocity of the user's ankle, the acceleration of the user's ankle, the angle of the user's ankle, the angular velocity of the user's ankle, the temperature of the user's ankle and the geomagnetism at the position of the user's ankle.

The sensor device 10F-1 is worn on the user's left foot. The sensor device 10F-2 is worn on the user's right foot. In this embodiment, the foot is the part extending from the user's ankle to the user's toes. The sensor device 10F may be a shoe-type wearable device. The sensor device 10F may be provided on or in a shoe. The sensor device 10F may be worn on the user's foot by using any method. The sensor device 10F may be worn on the user's foot by being incorporated into an anklet, a band, a friendship bracelet, an artificial fingernail, a tattoo sticker, a supporter, a cast, a sock, an insole, an artificial foot, a ring, an implant, and so on.

The sensor device 10F may be worn on the user's foot such that the x-axis of the local coordinate system based on the position of the sensor device 10F is parallel to the front-back direction of the foot as seen from the user's perspective, the y-axis of the local coordinate system is parallel to the left-right direction of the foot as seen from the user's perspective, and the z-axis of the local coordinate system is parallel to the up-down direction of the foot as seen from the user's perspective.

The sensor device 10F-1 detects sensor data representing the movement of the user's left foot. The sensor device 10F-2 detects sensor data representing the movement of the user's right ankle. The sensor data detected by the sensor device 10F includes, for example, at least any of the following: the velocity of the user's foot, the acceleration of the user's foot, the angle of the user's foot, the angular velocity of the user's foot, the temperature of the user's foot, and the geomagnetism at the position of the user's foot.

The electronic device 20 is carried by the user while walking, for example. The electronic device 20 is a mobile device such as a mobile phone, a smartphone, or a tablet.

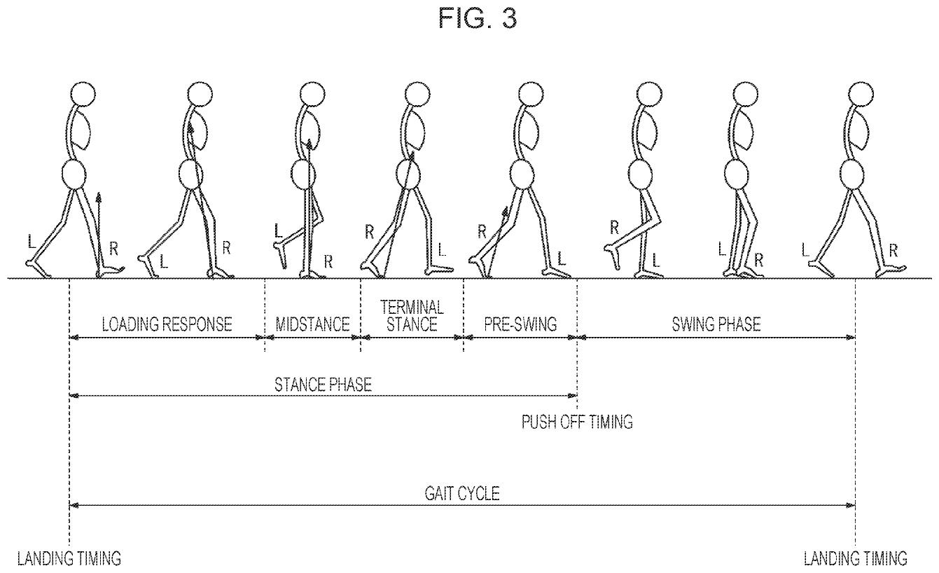

The electronic device 20 functions as an information processing device and acquires estimated values of the ground reaction force applied to the user based on sensor data detected by sensor device 10. Hereafter, the ground reaction force will be described together with the user's gait while referring to FIG. 3.

FIG. 3 illustrates the way in which the user walks, from the time when the user's right foot lands on the walking surface until the user's right foot lands on the walking surface again. In FIG. 3, the right leg of the user is denoted by the letter “R”. The left leg of the user is denoted by the letter “L”.

The ground reaction force is, for example, the reaction force generated from the contact area between the user's foot and the walking surface. In FIG. 3, the ground reaction force applied to the user's right foot is represented as an arrow.

Hereafter, one of the user's two feet will also be referred to as a “first foot”. In addition, the other of the user's two feet will also be referred to as a “second foot”. The gait cycle and so on will be described below while focusing on the first foot. In FIG. 3, the first foot is the right foot.

The gait cycle is the period of time from when the first foot lands on the walking surface until the next time the first foot lands on the walking surface. The starting point and the end point of the gait cycle correspond to the landing timing of the first foot. The landing timing is the timing at which the foot lands on the walking surface. In FIG. 3, the gait cycle is the period of time from when the user's right foot lands on the walking surface until the next time the user's right foot lands on the walking surface. The gait cycle includes a stance phase and a swing phase.

The stance phase is the period of time from when the first foot lands on the walking surface until when the first foot leaves the walking surface. The starting point of the stance phase is the landing timing of the first foot. The end point of the stance phase is when the first foot pushes off from the walking surface. The stance phase is the phase during which the first foot is in contact with the walking surface. During the stance phase, the first foot is in contact with the walking surface, resulting in a ground reaction force being applied to the first foot. In FIG. 3, the stance phase is the period of time from when the user's right foot lands on the ground until when the user's right foot leaves the ground.

The stance phase includes a loading response, a midstance, a terminal stance, and a pre-swing. During these periods, the ground reaction force changes in a variety of ways due to, for example changes in the area across which the first foot is in contact with the walking surface. If the ground reaction force can be estimated by varying the ground reaction force, these periods can be identified based on the estimated ground reaction force.

The loading response is the period during which a heel strike is performed on the walking surface from the heel of the first foot. In the loading response, a heel strike is performed on the walking surface from the heel of the first foot, and as a result, the ground reaction force increases. In the midstance, the user's body moves upward relative to the walking surface. The user's body moves upward relative to the walking surface, and as a result, the user's center of gravity is shifted upward relative to the walking surface by the greatest amount in the midstance. In the terminal stance, the user's body moves forward.

The swing phase is the period of time from when the first foot leaves the walking surface until when the first foot lands on the walking surface. In FIG. 3, this is the period of time from when the user's right foot leaves the walking surface until when the user's right foot lands on the walking surface. During the swing phase, the first foot is not in contact with the walking surface, and therefore no ground reaction force is applied to the first foot.

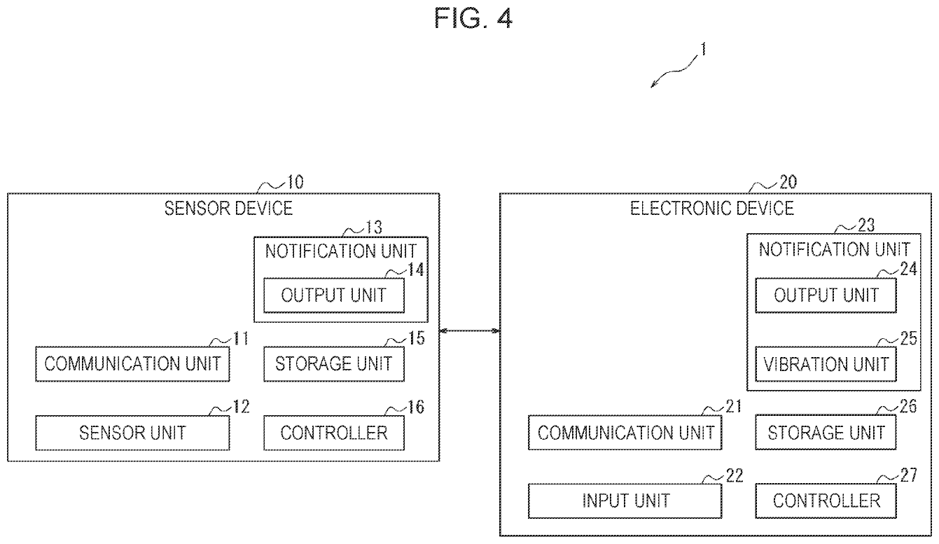

As illustrated in FIG. 4, the sensor device 10 includes a sensor unit 12. The sensor device 10 may further include a communication unit 11, a notification unit 13, a storage unit 15, and a controller 16. The sensor devices 10C to 10F do not need to include the notification unit 13.

The communication unit 11 includes at least one communication module capable of communicating with the electronic device 20 via a communication line. The communication module is a communication module that is compatible with communication standards of communication lines. The communication line standards are short-range wireless communication standards including, for example, Bluetooth (registered trademark), infrared, and NFC (Near Field Communication).

The sensor unit 12 includes any sensor depending on what sensor data is intended to be detected by the sensor device 10. The sensor unit 12 includes, for example, at least any of the following: a three-axis motion sensor, a three-axis acceleration sensor, a three-axis velocity sensor, a three-axis gyro sensor, a three-axis magnetometer, a temperature sensor, an inertial measurement unit (IMU), and a camera. When the sensor unit 12 includes a camera, the camera can detect the movement of a body part of the user by analyzing images of the body part captured by the camera.

When the sensor unit 12 includes an accelerometer and a magnetometer, data detected by each of the accelerometer and magnetometer may be used to calculate the initial angle of a body part to be detected by the sensor device 10. The data detected by each of the accelerometer and the magnetometer may be used to correct the data of the angle detected by the sensor device 10.

When the sensor unit 12 includes a gyro sensor, the angle of the body part to be detected by the sensor device 10 may be calculated by integrating the angular velocity detected by the gyro sensor over time.

The notification unit 13 reports information. In this embodiment, the notification unit 13 includes an output unit 14. However the notification unit 13 is not limited to the output unit 14. The notification unit 13 may include any component capable of outputting information.

The output unit 14 can output data. The output unit 14 includes at least one output interface capable of outputting data. The output interface is, for example, a display or a speaker. The display is, for example, an LCD (Liquid Crystal Display) or an organic EL (ElectroLuminescence) display.

If the output unit 14 is included in the sensor device 10A, the output unit 14 may include a speaker. If the output unit 14 is included in the sensor device 10B, the output unit 14 may include a display.

The storage unit 15 includes at least one semiconductor memory, at least one magnetic memory, at least one optical memory, or a combination of at least two of these types of memories. A semiconductor memory is, for example, a RAM (random access memory) or a ROM (read only memory). A RAM is for example, a SRAM (static random access memory) or a DRAM (dynamic random access memory). A ROM is, for example, an EEPROM (electrically erasable programmable read only memory). The storage unit 15 may function as a main storage device, an auxiliary storage device, or a cache memory. The storage unit 15 stores data used in operation of the sensor device 10 and data obtained by operation of the sensor device 10. For example, the storage unit 15 stores system programs, application programs, and embedded software.

The controller 16 includes at least one processor, at least one dedicated circuit, or a combination thereof. The processor can be a general-purpose processor such as a CPU (central processing unit) or a GPU (graphics processing unit), or a dedicated processor specialized for particular processing. A dedicated circuit is, for example, a FPGA (field-programmable gate array) or an ASIC (application specific integrated circuit). The controller 16 executes processing relating to operation of the sensor device 10 while controlling the various parts of the sensor device 10.

The controller 16 receives a signal instructing the start of data detection from the electronic device 20 via the communication unit 11. Upon receiving this signal, the controller 16 starts data detection. For example, the controller 16 acquires data detected by the sensor unit 12 from the sensor unit 12. The controller 16 transmits the acquired data, as sensor data, to the electronic device 20 via the communication unit 11. The signal instructing the start of data detection is transmitted from the electronic device 20 to multiple sensor devices 10 as a broadcast signal. A signal instructing the multiple sensor devices 10 to start data detection is transmitted as a broadcast signal to the multiple sensor devices 10 so that multiple sensor devices 10 can start data detection simultaneously.

The controller 16 acquires data from the sensor unit 12 at a preset time interval and transmits the acquired data as sensor data via the communication unit 11. The time interval may be set based on the walking speed of a typical user, for example. The same time interval may be used for each of the multiple sensor devices 10. This time interval being the same for the multiple sensor devices 10 allows the timings at which the multiple sensor devices 10 detect data to be synchronized.

As illustrated in FIG. 4, the electronic device 20 includes a communication unit 21, an input unit 22, a notification unit 23, a storage unit 26, and a controller 27.

The communication unit 21 includes at least one communication module capable of communicating with the sensor device 10 via a communication line. The communication module is at least one communication module that is compatible with communication standards of communication lines. The communication line standards are short-range wireless communication standards including, for example, Bluetooth (registered trademark), infrared, and NFC.

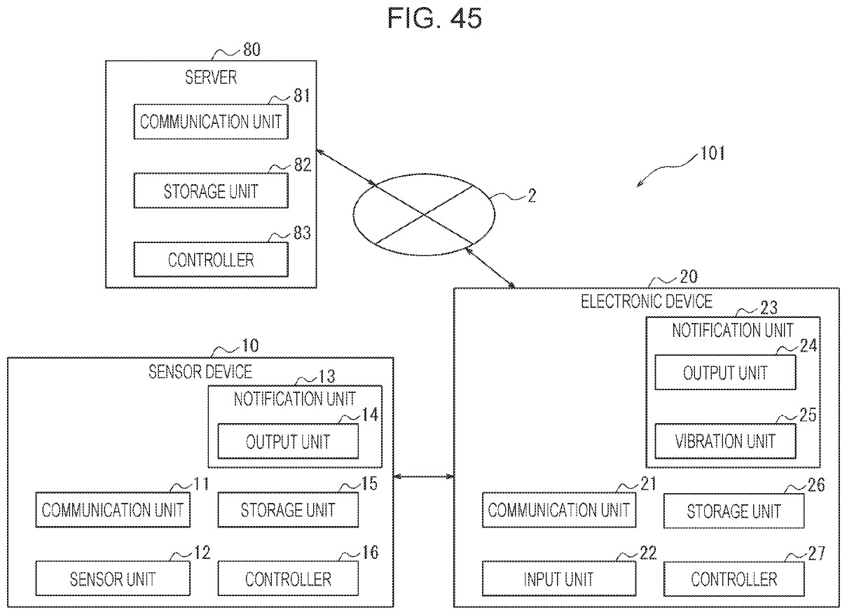

The communication unit 21 may further include at least one communication module that can connect to a network 2 as illustrated in FIG. 45 described later. The communication module is, for example, a communication module compatible with mobile communication standards such as LTE (Long Term Evolution), 4G (4th Generation) or 5G (5th Generation).

The input unit 22 is capable of receiving input from a user. The input unit 22 includes at least one input interface capable of receiving input from a user. The input interface takes the form of, for example, physical keys, capacitive keys, a pointing device, a touch screen integrated with the display, or a microphone.

The notification unit 23 reports information. In this embodiment, the notification unit 23 includes an output unit 24 and a vibration unit 25. However, the notification unit 23 is not limited to the output unit 24 and the vibration unit 25. The notification unit 23 may include any component capable of outputting information. The output unit 24 and vibration unit 25 may be mounted in the electronic device 20 or disposed in the vicinity of any of the sensor devices 10B to 10F.

The output unit 24 is capable of outputting data. The output unit 24 includes at least one output interface capable of outputting data. The output interface is, for example, a display or a speaker. The display is, for example, an LCD or organic EL display.

The vibration unit 25 is capable of making the electronic device 20 vibrate. The vibration unit 25 includes a vibration element. The vibration element is, for example, a piezoelectric element.

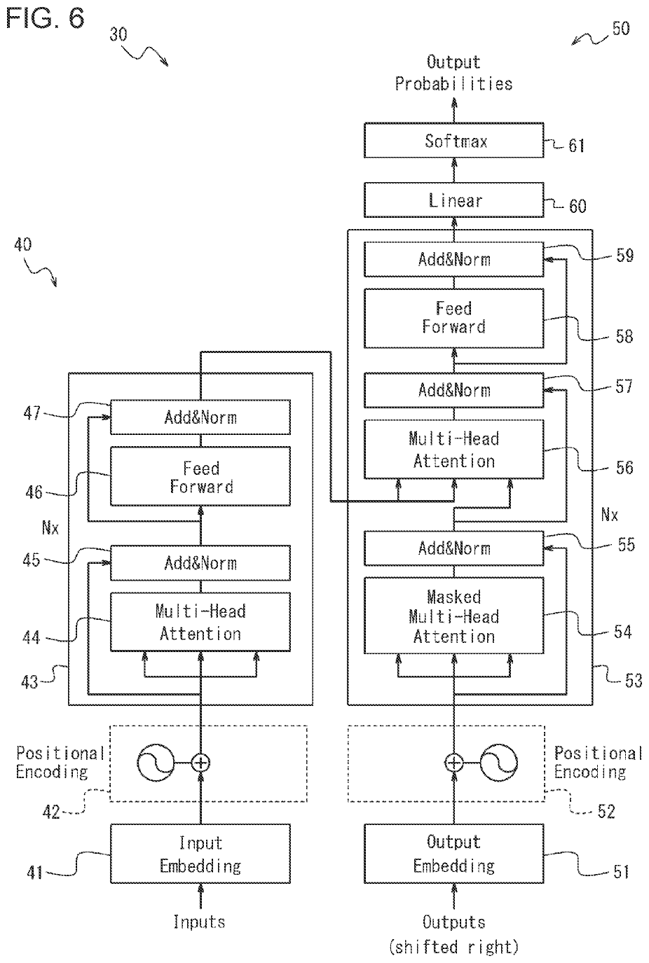

The storage unit 26 includes at least one semiconductor memory, at least one magnetic memory, at least one optical memory, or a combination of at least two of these types of memories. The semiconductor memory is, for example, a RAM or a ROM. The RAM is, for example, an SRAM or a DRAM. The ROM is, for example, an EEPROM. The storage unit 26 may function as a main storage device, an auxiliary storage device, or a cache memory. The storage unit 26 stores data used in operation of the electronic device 20 and data obtained by operation of the electronic device 20. For example, the storage unit 26 stores system programs, application programs, and embedded software. For example, the storage unit 26 stores data of a transformer 30 as illustrated in FIG. 6 described below and data used by the transformer 30.

The controller 27 includes at least one processor, at least one dedicated circuit, or a combination thereof. The processor can be a general-purpose processor such as a CPU or a GPU, or a dedicated processor specialized for particular processing. The dedicated circuit is, for example, an FPGA or an ASIC. The controller 27 executes processing relating to operation of the electronic device 20 while controlling the various parts of the electronic device 20. The controller 27 may perform the processing to be performed by the transformer 30 as illustrated in FIG. 6 described below.

The controller 27 accepts an input instructing execution of ground reaction force estimation processing via the input unit 22. This input is an input that causes the electronic device 20 to perform the ground reaction force estimation processing. This input is, for example, input from the input unit 22 by the user wearing the sensor device 10. The user inputs this input via the input unit 22, for example, before starting to walk. The controller 27 may accept at least any of inputs indicating the user's weight and the user's height via the input unit 22, along with the input instructing execution of the ground reaction force estimation processing. When at least any of the inputs indicating the user's weight and the user's height are received via the input unit 22, the controller 27 may store the received information on the user's weight and height in the storage unit 26.

When the controller 27 receives an input instructing execution of the ground reaction force estimation processing via the input unit 22, the controller 27 transmits a signal instructing the start of data detection as a broadcast signal to the multiple sensor devices 10 via the communication unit 21. After the signal instructing the start of data detection has been transmitted to the multiple sensor devices 10, sensor data is transmitted to the electronic device 20 from at least one of the sensor devices 10.

The controller 27 receives sensor data from at least one sensor device 10 via the communication unit 21. The controller 27 acquires the sensor data from the sensor device 10 by receiving the sensor data from the sensor device 10.

The controller 27 acquires estimated values of the ground reaction force acting on the user by using the sensor data and a trained model. When using sensor data of the global coordinate system, the controller 27 may acquire sensor data of the global coordinate system by performing a coordinate transformation on the sensor data of the local coordinate system acquired from the sensor device 10.

The trained model is, for example, generated by machine learning so as to output estimated valued of the ground reaction force when input with sensor data. In this embodiment, the controller 27 uses the transformer described in “Attention Is All You Need” by Ashish Vaswani et al, Jun. 12, 2017, arXiv: 1706.03762v5 [cs. CL]” as the trained model. A transformer can process time series data. However, the trained model is not limited to a transformer. The controller 27 may use a trained model generated by machine learning based on any machine learning algorithm. The configuration of the transformer is described later.

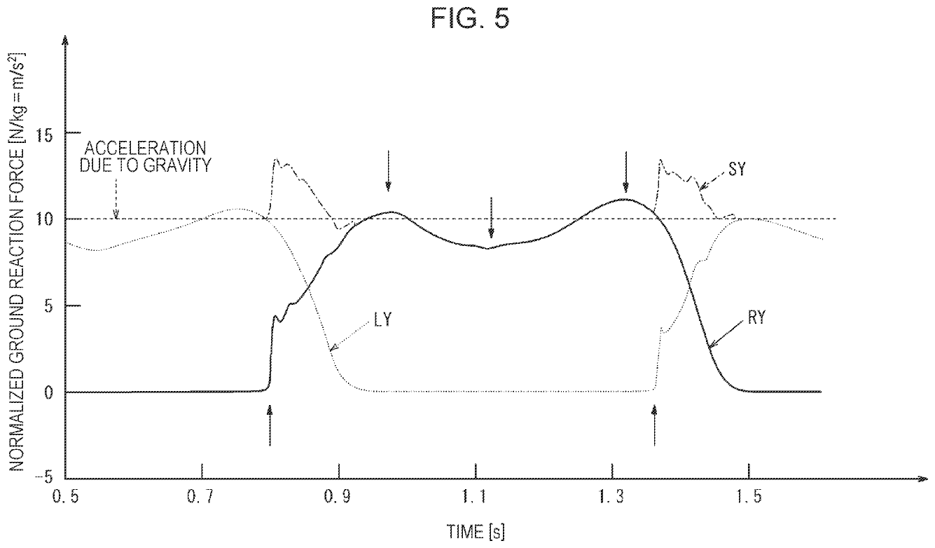

The controller 27 acquires estimated values of the normalized ground reaction force as illustrated in FIG. 5, for example, by using the sensor data and the transformer. The normalized ground reaction force is obtained by normalizing the ground reaction force applied to the user. In this embodiment, the normalized ground reaction force is normalized by dividing the ground reaction force applied to the user by the user's weight. The normalized ground reaction force is the sum of the acceleration due to gravity of 9.80 [m/s²] and the acceleration of the user's motion. As discussed below, the transformer can be trained to output estimated values of the normalized ground reaction force when input with sensor data.

FIG. 5 illustrates a graph of the estimated values of the normalized ground reaction force. The horizontal axis in FIG. 5 represents time[s]. The vertical axis in FIG. 5 represents normalized ground reaction force [N/kg]. FIG. 5 also illustrates the acceleration due to gravity, along with the estimated values of the normalized ground reaction force. A normalized ground reaction force LY is the normalized ground reaction force along the Y-axis of the global coordinate system out of the normalized ground reaction force acting on the user's left foot. A normalized ground reaction force RY is the normalized ground reaction force along the Y-axis of the global coordinate system out of the normalized ground reaction force acting on the user's right foot. A normalized ground reaction force SY is the sum of the normalized ground reaction force LY and the normalized ground reaction force RY. The direction from top to bottom from the perspective of the user is taken to be the positive direction of the normalized ground reaction force along the Y-axis.

If an input indicating the user's weight is received via the input unit 22, the controller 27 may calculate a calculated value of the ground reaction force by multiplying the estimated value of the normalized ground reaction force by the user's weight. Here, in this specification, when an estimated value of the normalized ground reaction force and a calculated value of the ground reaction force calculated from an estimated value of the normalized ground reaction force are not particularly distinguished from each other, these values are also collectively referred to as “estimated value of the ground reaction force”.

The controller 27 may cause the notification unit 23 to report the estimated values of the ground reaction force. For example, the controller 27 may display information depicting the estimated values of the ground reaction force as illustrated in FIG. 5 on a display of the output unit 24. Alternatively, the controller 27 may cause a speaker of the output unit 24 to output information indicating the estimated values of the ground reaction force as sound. As described later, the controller 27 may cause the sensor device 10 to report the estimated values of the ground reaction force. As described later, the controller 27 may acquire any information about the user's gait based on the estimated values of the ground reaction force, or may evaluate the user's gait.

### [Configuration of Transformer]

Hereafter, the transformer will be described while referring to FIG. 6. The transformer 30, as illustrated in FIG. 6, can be trained to output time series data of estimated values of the normalized ground reaction force applied to the user's right foot or left foot when input with sensor data in multiple time series. The transformer 30 may be trained to output time series data of estimated values of the normalized ground reaction force for all of the X, Y, and Z axes, or may be trained to output time series data of estimated values of the normalized ground reaction force for any one or more of the X, Y, and Z axes. The time range and time interval of the sensor data in the time series input to the transformer 30 may be set in accordance with the desired estimation accuracy and so on.

As illustrated in FIG. 6, the transformer 30 includes an encoder 40 and a decoder 50. The encoder 40 includes a functional unit 41, a functional unit 42, and an N-stage layer 43. The layer 43 includes a functional unit 44, a functional unit 45, a functional unit 46, and a functional unit 47. The decoder 50 includes a functional unit 51, a functional unit 52, an N-stage layer 53, a functional unit 60, and a functional unit 61. The layer 53 includes a functional unit 54, a functional unit 55, a functional unit 56, a functional unit 57, a functional unit 58, and a functional unit 59. The number of stages of the N-stage layer 43 of the encoder 40 and the number of stages of the N-stage layer 53 of the decoder 50 are identical, i.e., N stages.

The functional unit 41 is also referred to as “Input Embedding”. An array of sensor data along a plurality of time series is input to the functional unit 41. For example, if the sensor data at time ti(0≤i≤n) is denoted as “Dui”, the array of sensor data input to the functional unit 41 is represented as (Dₜ₀, Dₜ₁, . . . , Dₜₙ). An array consisting of multiple types of sensor data may be input to the functional unit 41. For example, if two different pieces of sensor data at time ti(0≤i≤n) are denoted as “Daₜᵢ” and “Dbₜᵢ” respectively, the array of sensor data input to the functional unit 41 is represented as (Daₜ₀, Daₜ₁, . . . , Daₜₙ, Dbₜ₀, Dₜ₁, . . . , Dbₜₙ).

The functional unit 41 converts each element of the array of input sensor data into a multidimensional vector, and in this way, generates a distributed representation vector. The number of dimensions of the multidimensional vector may be set in advance.

The functional unit 42 is also referred to as “Positional Encoding”. The functional unit 42 assigns position information to the distributed representation vector.

The functional unit 42 calculates and adds position information to each element of the distributed representation vector. The position information represents the position of each element of the distributed representation vector in the array of sensor data input to the functional unit 41 and represents the position in the element array of the distributed representation vector. The functional unit 42 calculates position information PE of the (2xi)th element in the array of elements of the distributed representation vector using Eq. (1). The functional unit 42 calculates position information PE of the (2xi+1)th element in the array of elements of the distributed representation vector using Eq. (2).

$$\begin{matrix}[ Math . 1 ] & \\ {PE}_{( pos , 2 i )} = sin ( pos / 1000 {0}^{2 i / {d}_{model}} ) & Eq . ( 1 )\end{matrix}$$

$$\begin{matrix}[ Math . 2 ] & \\ {PE}_{( pos , 2 i + 1 )} = cos ( pos / 1000 {0}^{2 i / {d}_{model}} ) & Eq . ( 2 )\end{matrix}$$

In Eq. (1) and Eq. (2), pos is the position, in the array of sensor data input to the functional unit 41, of the elements of the distributed representation vector. d_{model} is the number of dimensions of the distributed representation vector.

In the N-stage layer 43, a vector assigned with position information and having a distributed representation is input from the functional unit 42 to the first stage of the layer 43. The second and subsequent stages of the layer 43 are input with vectors from the previous stages of the layer 43.

The functional unit 44 is also referred to as “Multi-Head Attention”. A Q (Query) vector, a K (Key) vector, and a V (Value) vector are input to the functional unit 44. The Q vector is obtained by multiplying the vector input to the layer 43 by a weight matrix WQ. The K vector is obtained by multiplying the vector input to the layer 43 by a weight matrix WK. The V vector is obtained by multiplying the vector input to layer 43 by a weight matrix WV. The transformer 30 learns the weight matrix WQ, the weight matrix WK, and the weight matrix WV during training.

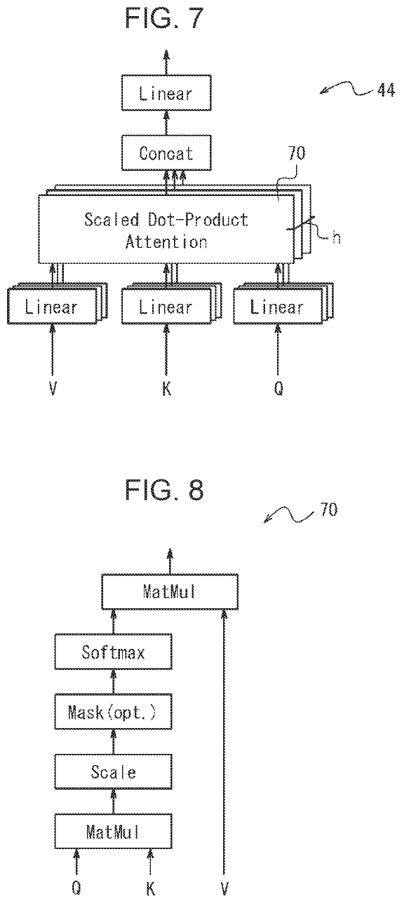

The functional unit 44 includes h functional units 70 and functional units “Linear” and “Contact” as illustrated in FIG. 7. The functional units 70 are also referred to as “Scaled Dot-Product Attention”. The Q-vector, K-vector, and V-vector, divided into h pieces, are input to the functional units 70.

Each functional unit 70 includes functional units “MatMul”, “Scale”, “Mask (opt.)”, and “Softmax”, as illustrated in FIG. 8. The functional unit 70 calculates the Scaled Dot-Product Attention using the Q-vector, K-vector, and V-vector and Eq. (3).

$$\begin{matrix}[ Math . 3 ] & \\ Attention ( Q , K , V ) = softmax ( \frac{{QK}^{T}}{\sqrt{{d}_{k}}} ) V & Eq . ( 3 )\end{matrix}$$

In Eq. (3), dₖ is the number of dimensions of the Q vector and the K vector.

The functional unit 44 calculates Multi-Head Attention when the Scaled Dot-Product Attention is calculated by the h functional units 70 as illustrated in FIG. 7. The functional unit 44 calculates the Multi-Head Attention using Eq. (4).

$$\begin{matrix}[ Math . 4 ] & \\ MutiHead ( Q , K , V ) = Concat ( {head}_{1} , … , {head}_{h} ) {W}^{O} & Eq . ( 4 )\end{matrix}$$

$$Here ,$$

$${head}_{i} = Attention ( {QW}_{i}^{Q} , {KW}_{i}^{K} , {VW}_{i}^{V} )$$

$${W}_{i}^{Q} ∈ {ℝ}^{{d}_{model} \times {d}_{k}} ,$$

$${W}_{i}^{K} ∈ {ℝ}^{{d}_{model} \times {d}_{k}} ,$$

$${W}_{i}^{V} ∈ {ℝ}^{{d}_{model} \times {d}_{v}} ,$$

$${W}^{O} ∈ {ℝ}^{{hd}_{v} \times {d}_{model}}$$

In Eq. (4), dₖ is the number of dimensions of the Q vector and the K vector. dᵥ is the number of dimensions of the V vector.

The Multi-Head Attention calculated by the functional unit 44 is input to functional unit 45 as illustrated in FIG. 6.

The functional unit 45 is also referred to as “Add&Norm”. The functional unit 45 adds the Multi-Head Attention calculated by the functional unit 44 to the vector input to the layer 43 and normalizes the resulting vector. The functional unit 45 inputs the normalized vector to the functional unit 46.

The functional unit 46 is also referred to as “Position-wise Feed-Forward Networks”. The functional unit 46 generates an output by using an activation function such as an ReLU (Rectified Linear Unit) and a vector input from the functional unit 45. The functional unit 46 uses a different FFN (Feed-Forward Network) for each position in the element array of the sensor data along the time series before vectorization, i.e., the sensor data along the time series input to the functional unit 41. If the vector input from the functional unit 45 to the functional unit 46 is denoted as “x,” then the functional unit 46 generates an output FFN(x) according to Eq. (5).

$$\begin{matrix}[ Math . 5 ] & \\ FFN ( x ) = max ( 0 , {xW}_{1} + {b}_{1} ) {W}_{2} + {b}_{2} & Eq . ( 5 )\end{matrix}$$

In Eq. (5), W₁ and W₂ are coefficients. b₁ and b₂ are biases. W₁ and W₂ and b₁ and b₂ may be different for each position in the element array of sensor data along the time series before vectorization.

The functional unit 47 is also referred to as “Add&Norm”. The functional unit 47 adds an output generated by the functional unit 46 to the vector output from the functional unit 45 and normalizes the resulting vector.

The functional unit 51 is also referred to as “Input Embedding”. The functional unit 51 is input with the time series data of the estimated values of the normalized ground reaction force output by the decoder 50 in the immediately previous processing. When the decoder 50 estimates the ground reaction force data for the first time, the functional unit 51 may be input with preset data such as dummy data. In the same or a similar manner to the functional unit 41, the functional unit 51 converts each element of the input time series data into a multidimensional vector, and thereby generates a distributed representation vector. In the same or a similar manner to the functional unit 41, the number of dimensions of the multidimensional vector may be predetermined.

The functional unit 52 is also referred to as “Positional Encoding”. The functional unit 52 assigns position information to the distributed representation vector in the same or a similar manner to the functional unit 42. In other words, the functional unit 52 calculates and adds position information for each element of the distributed representation vector. The position information represents the position of each element of the distributed representation vector in the array of time series data input to the functional unit 51 and in the element array of the distributed representation vector.

In the N-stage layer 53, a vector assigned with position information and having a distributed representation is input from the functional unit 52 to the first stage of the layer 53. The second and subsequent stages of the layer 53 are input with vectors from the previous stage of the layer 53.

The functional unit 54 is also referred to as “Masked Multi-Head Attention”. The Q-vector, the K-vector, and the V-vector are input to the functional unit 54 in an identical or similar manner to the functional unit 44. The Q vector, the K vector, and the V vector are obtained by multiplying vectors input to the layer 53 by the same weight matrix or different weight matrices. The transformer 30 learns these weight matrices during training. The functional unit 54 calculates the Multi-Head Attention using the input Q-vector, K-vector, and V-vector, in the same or a similar manner to the functional unit 44.

Here, the functional unit 54 is input, one time, with time series data of the normalized ground reaction force, which is the correct solution, during training of the transformer 30. During training of the transformer 30, the functional unit 54 masks the data, in the time series data of the normalized ground reaction force, at times from the time at which estimation is to be performed by the decoder 50.

The functional unit 55 is also referred to as “Add&Norm”. The functional unit 55 adds the Multi-Head Attention calculated by the functional unit 54 to the vector input to the layer 53 and normalizes the resulting vector.

The functional unit 56 is also referred to as “Multi-Head Attention”. A Q-vector, a K-vector, and a V-vector are input to the functional unit 56. The Q vector is the vector input to the functional unit 56 by the functional unit 55 after normalization. The K-vector and the V-vector are obtained by multiplying a vector output from the final stage of the layer 43 of the encoder 40 by the same or different weight matrices. The functional unit 56 calculates the Multi-Head Attention using the input Q-vector, K-vector, and V-vector, in the same or a similar manner to the functional unit 44.

The functional unit 57 is also referred to as “Add&Norm”. The functional unit 57 adds the Multi-Head Attention calculated by the functional unit 56 to the vector output by the functional unit 55 and normalizes the resulting vector.

The functional unit 58 is also referred to as “Position-wise Feed-Forward Networks”. The functional unit 58 generates an output by using an activation function such as ReLU and a vector input from the functional unit 57 in an identical or similar manner to the functional unit 46.

The functional unit 59 is also referred to as “Add&Norm”. The functional unit 59 adds an output generated by the functional unit 58 to the vector output from the functional unit 57 and normalizes the resulting vector.

The functional unit 60 is also referred to as “Linear”. The functional unit 61 is also referred to as “SoftMax”. The output of the final stage of the layer 53 is output from the decoder 50 as data of estimated values of the ground reaction force after being normalized and so on by the functional unit 60 and the functional unit 61.

### [Combinations of Sensor Data]

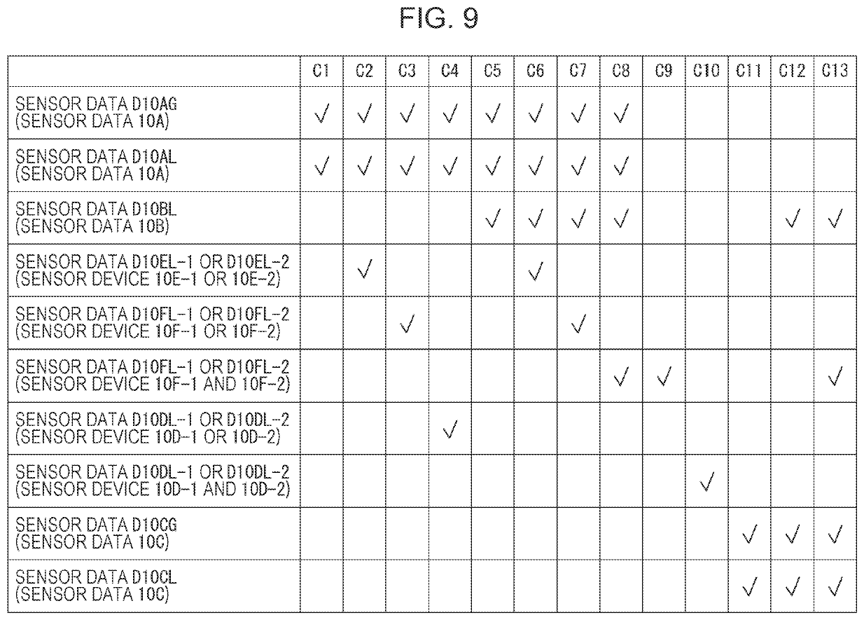

The controller 27 may use a transformer trained on one type of sensor data or a transformer trained on a combination of multiple types of sensor data. Combinations of multiple types of sensor data are, for example, cases C1, C2, C3, C4, C5, C6, C7, C8, C9, C10, C11, C12, and C13, as illustrated in FIG. 9.

FIG. 9 illustrates examples of combinations of sensor data. The cases C1 to C13 are examples of combinations of sensor data. The controller 27 may select any of the cases C1 to C13 in accordance with the type of sensor device 10 that transmitted the sensor data to the electronic device 20. The data of the transformer 30 used in the cases C1 to C13 may be stored in storage unit 26 in association with the cases C1 to C13, respectively. The controller 27 acquires estimated values of the normalized ground reaction force by inputting the sensor data of the selected any one of the cases C1 to C13 to the transformer 30 corresponding to the selected any one of the cases C1 to C13.

### <Case C1>

The controller 27 may select the case C1 if the sensor devices 10 that transmitted sensor data to the electronic device 20 include the sensor device 10A.

In the case C1, sensor data representing the movement of the user's head is used. In the case C1, sensor data D10AG and sensor data D10AL are used.

The sensor data D10AG is sensor data representing the movement of the user's head in the global coordinate system. The sensor data D10AG includes velocity data and acceleration data of the user's head with respect to the x-axis, velocity data and acceleration data of the user's head with respect to the y-axis, and velocity data and acceleration data of the user's head with respect to the z-axis in the global coordinate system. The controller 27 acquires the sensor data D10AG by performing a coordinate transformation on the sensor data in the local coordinate system acquired from the sensor device 10A.

The sensor data D10AL is sensor data representing the movement of the user's head in the local coordinate system with respect to the position of the sensor device 10A. The sensor data D10AL includes velocity data and acceleration data of the user's head with respect to the x-axis, velocity data and acceleration data of the user's head on the y-axis, and velocity data and acceleration data of the user's head with respect to the z-axis in the local coordinate system. The controller 27 acquires the sensor data D10AL from the sensor device 10A.

### <Case C2>

The controller 27 may select the case C2 if the sensor devices 10 that transmitted sensor data to the electronic device 20 include the sensor device 10A and the sensor device 10E-1 or the sensor device 10E-2.

In the case C2, sensor data representing the movement of the user's head and sensor data representing the movement of either one of the user's two ankles are used. In the case C2, the sensor data D10AG, the sensor data D10AL, and sensor data D10EL-1 or sensor data D10EL-2 are used.

The sensor data D10EL-1 is sensor data representing the movement of the user's left ankle in the local coordinate system with respect to the position of the sensor device 10E-1. The sensor data D10EL-1 includes velocity data and acceleration data of the user's left ankle with respect to the x-axis, velocity data and acceleration data of the user's left ankle with respect to the y-axis, and velocity data and acceleration data of the user's left ankle with respect to the z-axis in the local coordinate system. The controller 27 acquires the sensor data D10EL-1 from the sensor device 10E-1.

The sensor data D10EL-2 is sensor data representing the movement of the user's right ankle in the local coordinate system with respect to the position of the sensor device 10E-2. The sensor data D10EL-2 includes velocity data and acceleration data of the user's right ankle with respect to the x-axis, velocity data and acceleration data of the user's right ankle with respect to the y-axis, and velocity data and acceleration data of the user's right ankle with respect to the z-axis in the local coordinate system. The controller 27 acquires the sensor data D10EL-2 from the sensor device 10E-2.

### <Case C3>

The controller 27 may select the case C3 if the sensor devices 10 that transmitted sensor data to the electronic device 20 include the sensor device 10A and the sensor device 10F-1 or the sensor device 10F-2.

In the case C3, sensor data representing the movement of the user's head and sensor data representing the movement of either one of the user's two feet are used. In the case C3, the sensor data D10AG, the sensor data D10AL, and sensor data D10FL-1 or sensor data D10FL-2 are used.

The sensor data D10FL-1 is sensor data representing the movement of the user's left foot in the local coordinate system with respect to the position of the sensor device 10F-1. The sensor data D10FL-1 includes velocity data and acceleration data of the user's left foot with respect to the x-axis, velocity data and acceleration data of the user's left foot with respect to the y-axis, and velocity data and acceleration data of the user's left foot with respect to the z-axis in the local coordinate system. The controller 27 acquires the sensor data D10FL-1 from the sensor device 10F-1.

The sensor data D10FL-2 is sensor data representing the movement of the user's right foot in the local coordinate system with respect to the position of the sensor device 10F-2. The sensor data D10FL-2 includes velocity data and acceleration data of the user's right foot with respect to the x-axis, velocity data and acceleration data of the user's right foot with respect to the y-axis, and velocity data and acceleration data of the user's right foot with respect to the z-axis in the local coordinate system. The controller 27 acquires the sensor data D10FL-2 from the sensor device 10F-2.

### <Case C4>

The controller 27 may select the case C4 if the sensor devices 10 that transmitted sensor data to the electronic device 20 include the sensor device 10A and the sensor device 10D-1 or the sensor device 10D-2.

In the case C4, sensor data representing the movement of the user's head and sensor data representing the movement of either one of the user's two thighs are used. In the case C4, the sensor data D10AG, the sensor data D10AL, and sensor data D10DL-1 or sensor data D10DL-2 are used.

The sensor data D10DL-1 is sensor data representing the movement of the user's left thigh in the local coordinate system with respect to the position of the sensor device 10D-1. The sensor data D10DL-1 includes velocity data and acceleration data of the user's left thigh with respect to the x-axis, velocity data and acceleration data of the user's left thigh with respect to the y-axis, and velocity data and acceleration data of the user's left thigh with respect to the z-axis in the local coordinate system. The controller 27 acquires the sensor data D10DL-1 from the sensor device 10D-1.

The sensor data D10DL-2 is sensor data representing the movement of the user's right thigh in the local coordinate system with respect to the position of the sensor device 10D-2. The sensor data D10DL-2 includes velocity data and acceleration data of the user's right thigh with respect to the x-axis, velocity data and acceleration data of the user's right thigh with respect to the y-axis, and velocity data and acceleration data of the user's right thigh with respect to the z-axis in the local coordinate system. The controller 27 acquires the sensor data D10DL-2 from the sensor device 10D-2.

### <Case C5>

The controller 27 may select the case C5 if the sensor devices 10 that transmitted sensor data to the electronic device 20 include the sensor device 10A and the sensor device 10B.

In the case C5, sensor data representing the movement of the user's head and sensor data representing the movement of either one of the user's two wrists are used. In the case C5, the sensor data D10AG, the sensor data D10AL, and sensor data D10BL are used.

The sensor data D10BL is sensor data representing the movement of the user's wrist in the local coordinate system with respect to the position of the sensor device 10B. The sensor data D10BL includes velocity data and acceleration data of the user's wrist with respect to the x-axis, velocity data and acceleration data of the user's wrist on the y-axis, and velocity data and acceleration data of the user's wrist with respect to the z-axis in the local coordinate system. The controller 27 acquires the sensor data D10BL from the sensor device 10B.

### <Case C6>

The controller 27 may select the case C6 if the sensor devices 10 that transmitted sensor data to the electronic device 20 include the sensor devices 10A and 10B and the sensor device 10E-1 or the sensor device 10E-2.

In the case C6, sensor data representing the movement of the user's head, sensor data representing the movement of either one of the user's two wrists, and sensor data representing the movement of either one of the user's two ankles are used. In the case C6, the sensor data D10AG, the sensor data D10AL, the sensor data D10BL, and the sensor data D10EL-1 or the sensor data D10EL-2 are used.

### <Case C7>

The controller 27 may select the case C7 if the sensor devices 10 that transmitted sensor data to the electronic device 20 include the sensor devices 10A and 10B, and the sensor device 10F-1 or the sensor device 10F-2.

In the case C7, sensor data representing the movement of the user's head, sensor data representing the movement of either one of the user's two wrists, and sensor data representing the movement of either one of the user's two feet are used. In the case C7, the sensor data D10AG, the sensor data D10AL, the sensor data D10BL, and the sensor data D10FL-1 or the sensor data D10FL-2 are used.

### <Case C8>

The controller 27 may select the case C8 if the sensor devices 10 that transmitted sensor data to the electronic device 20 include the sensor devices 10A, 10B, 10F-1, and 10F-2.

In the case C8, sensor data representing the movement of the user's head, sensor data representing the movement of either one of the user's two wrists, and sensor data representing the movement of each of the user's two feet are used. In the case C8, the sensor data D10AG, the sensor data D10AL, the sensor data D10BL, the sensor data D10FL-1, and the sensor data D10FL-2 are used.

### <Case C9>

The controller 27 may select the case C9 if the sensor devices 10 that transmitted sensor data to the electronic device 20 include the sensor device 10F-1 and the sensor device 10F-2.

In the case C9, sensor data representing the movement of each of the user's two feet is used. In the case C9, the sensor data D10FL-1 and the sensor data D10FL-2 are used.

### <Case C10>

The controller 27 may select the case C10 if the sensor devices 10 that transmitted sensor data to the electronic device 20 include the sensor devices 10D-1 and 10D-2.

In the case C10, sensor data representing the movement of each of the user's two thighs is used. In the case C10, the sensor data D10DL-1 and the sensor data D10DL-2 are used.

The sensor data D10DL-1 is sensor data representing the movement of the user's left thigh in the local coordinate system with respect to the position of the sensor device 10D-1. The sensor data D10DL-1 includes velocity data and acceleration data of the user's left thigh with respect to the x-axis, velocity data and acceleration data of the user's left thigh with respect to the y-axis, and velocity data and acceleration data of the user's left thigh with respect to the z-axis in the local coordinate system. The controller 27 acquires the sensor data D10DL-1 from the sensor device 10D-1.

The sensor data D10DL-2 is sensor data representing the movement of the user's right thigh in the local coordinate system with respect to the position of the sensor device 10D-2. The sensor data D10DL-2 includes velocity data and acceleration data of the user's right thigh with respect to the x-axis, velocity data and acceleration data of the user's right thigh with respect to the y-axis, and velocity data and acceleration data of the user's right thigh with respect to the z-axis in the local coordinate system. The controller 27 acquires the sensor data D10DL-2 from the sensor device 10D-2.

### <Case C11>

The controller 27 may select the case C11 if the sensor devices 10 that transmitted sensor data to the electronic device 20 include the sensor device 10C.

In the case C11, sensor data representing the movement of the user's waist is used. In the case C11, sensor data D10CG and sensor data D10CL are used.

The sensor data D10CG is sensor data representing the movement of the user's waist in the global coordinate system. The sensor data D10CG includes velocity data and acceleration data of the user's waist with respect to the x-axis, velocity data and acceleration data of the user's waist with respect to the y-axis, and velocity data and acceleration data of the user's waist with respect to the z-axis in the global coordinate system. The controller 27 acquires the sensor data D10CG by performing a coordinate transformation on the sensor data in the local coordinate system acquired from the sensor device 10C.

The sensor data D10CL is sensor data representing the movement of the user's waist in the local coordinate system with respect to the position of the sensor device 10C. The sensor data D10CL includes velocity data and acceleration data of the user's waist with respect to the x-axis, velocity data and acceleration data of the user's waist with respect to the y-axis, and velocity data and acceleration data of the user's waist with respect to the z-axis in the local coordinate system. The controller 27 acquires the sensor data D10CL from the sensor device 10C.

### <Case C12>

The controller 27 may select the case C12 if the sensor devices 10 that transmitted sensor data to the electronic device 20 include the sensor device 10B and the sensor device 10C.

In the case C12, sensor data representing the movement of either one of the user's two wrists and sensor data representing the movement of the user's waist are used. In the case C12, the sensor data D10BL, the sensor data D10CG, and the sensor data D10CL are used.

### <Case C13>

The controller 27 may select the case C13 if the sensor devices 10 that transmitted sensor data to the electronic device 20 include the sensor devices 10B, 10F-1, 10F-2, and 10C.

In the case C13, sensor data representing the movement of either one of the user's two wrists, sensor data representing the movement of each of the user's two feet, and sensor data representing the movement of the user's waist are used. In the case C13, the sensor data D10BL, the sensor data D10FL-1, the sensor data D10FL-2, the sensor data D10CG, and the sensor data D10CL are used.

### [Generation and Evaluation of Transformer]

Next, generation and evaluation of a transformer will be described. A subject gait database was used in the generation of the transformer. As the subject gait database, data provided in “Kobayashi, Y., Hida, N., Nakajima, K., Fujimoto, M., Mochimaru, M., “2019: AIST Gait Database 2019,” [Online], [retrieved Aug. 20, 2021], Internet <https://unit.aist.go.jp/harc/ExPART/GDB2019_e.html>” was used. Gait data of multiple subjects are registered in this gait database. The gait data of a subject includes data representing the movement of the subject while walking and data on the ground reaction force applied to the subject while walking. The data representing the movement of the subject while walking were detected by a motion capture system. The data on the ground reaction force applied to the subject while walking was detected by a ground reaction force meter.

The transformer was generated by using, as training data, a dataset containing data on the ground reaction forces of multiple subjects detected by a ground reaction force meter and data representing the movements of the subjects while walking detected by a motion capture system.

In this embodiment, when generating the data set, normalized ground reaction forces were acquired by dividing the ground reaction force detected by the ground reaction force meter by the subject's body weight. Data corresponding to sensor data was acquired from the data representing the movements of the subjects detected by the motion capture system. A data set was generated by associating the normalized ground reaction forces with data corresponding to the sensor data. Data sets corresponding to the cases C1 to C13 described above with reference to FIG. 9 were generated. Training of the transformer was performed with the generated datasets. In the training of the transformer, the datasets were given about 10% noise in order to counteract over-training.

The inventors evaluated the trained transformer by using data sets that were not used in training of the transformer. The inventors obtained evaluation results for the cases C1 to C13 described above with reference to FIG. 9.

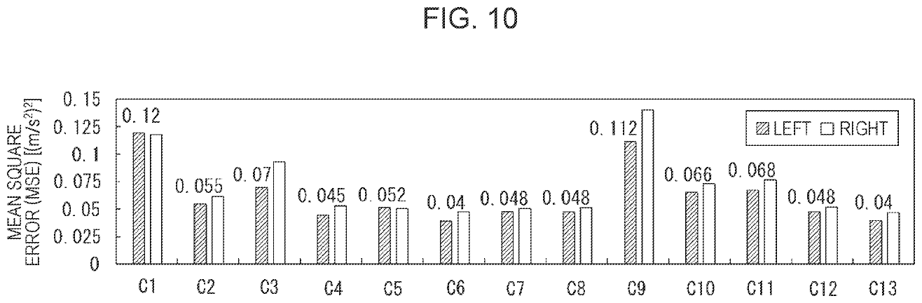

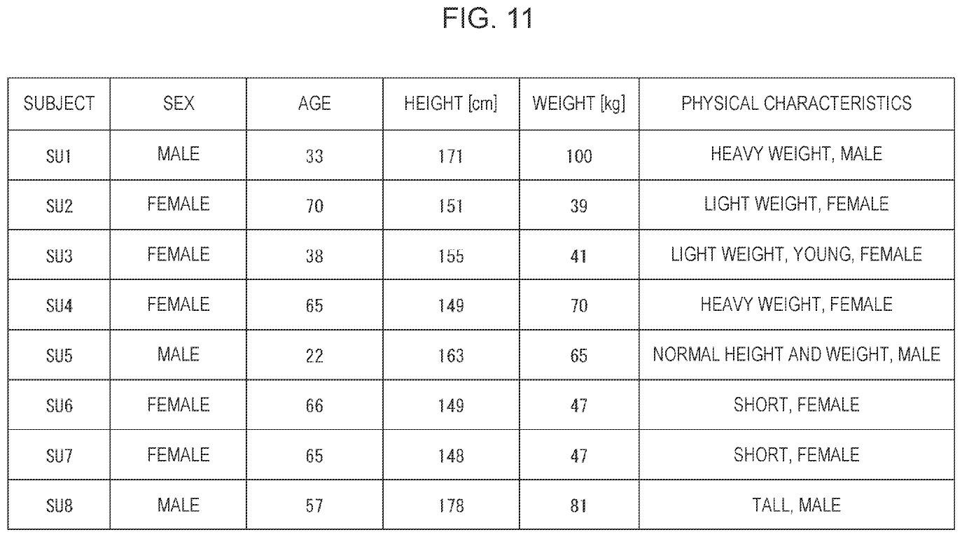

A graph of the evaluation results is illustrated in FIG. 10. FIG. 10 illustrates a bar chart of the Mean Squared Error (MSE) of the normalized ground reaction force for each of the cases C1 to C13 as evaluation results. The mean squared error data depicted in FIG. 10 were obtained from the subjects illustrated in FIG. 11, described below. The mean squared error was calculated based on the estimated value of the normalized ground reaction force produced by the transformer and the measured value of the normalized ground reaction force in the data set. In the bar chart, the left foot normalized ground reaction force evaluation results are shaded with hatching. In the bar chart, the right foot normalized ground reaction force evaluation results are illustrated in white. In FIG. 10, the numbers appended to the bars are the mean squared errors of the normalized ground reaction forces on the left foot. The mean squared error was calculated using Eq. (6) below.

$$\begin{matrix}[ Math . 6 ] & \\ {MSE}_{seq} = \frac{1}{n} \times \frac{1}{d} {\sum }_{j = 1}^{n} {\sum }_{i = 1}^{d} {( {a}_{i , j} - {b}_{i , j} )}^{2} & Eq . ( 6 )\end{matrix}$$

In Eq. (6), j corresponds to the X-axis, the Y-axis, and the Z-axis of the global coordinate system. d is the number of dimensions of the global coordinate system, i.e., 3. a_{i,j} is a measured value of the normalized ground reaction force of the data set. b_{i,j} is an estimated value of the normalized ground reaction force. n is the number of samples for the normalized ground reaction force.

As illustrated in FIG. 10, in the cases C1 to C13, the evaluation results of the normalized ground reaction force on the left foot and the evaluation results of the normalized ground reaction force on the right foot exhibited similar trends in terms of estimation accuracy. Since the estimation results for the normalized ground reaction force on the left foot and the estimation results for the normalized ground reaction force on the right foot exhibit similar trends in terms of estimation accuracy, hereafter, we will primarily focus on the mean squared error of the left foot normalized ground reaction force.

As illustrated in FIG. 10, in the case C1, the mean squared error of the normalized ground reaction force on the left foot was around 0.12 [(m/s²)²]. In the case C1, only sensor data representing the movement of the user's head, which is the farthest away from the user's feet, among the user's body parts is used. The estimation results of the case C1 demonstrate that the ground reaction force can be estimated with some degree of accuracy even when only sensor data representing the movement of the user's head is used. The reason for this is presumably that the movement of the user in the up-down direction while walking is reflected in the movement of the head.

As illustrated in FIG. 10, the mean squared errors in the cases C2 to C8 were smaller than the mean squared error in the case C1. In other words, the estimation accuracy of the ground reaction force in the cases C2 to C8 was improved compared to that in the case C1. In the cases C2 to C8, sensor data representing the movement of at least any of the user's wrists, ankles, feet, and thighs is used in addition to the sensor data representing the movement of the user's head, as described above. In other words, in the cases C2 to C8, sensor data representing the movement of the user's limbs, including at least any of the user's wrists, ankles, feet, and thighs, are used in addition to sensor data representing the movement of the user's torso including the user's head. Sensor data representing the movement of the user's limbs and sensor data representing the movement of the user's torso have significantly different patterns from each other within a single gait cycle. For example, due to the left-right symmetry of the user's body, the sensor data representing the movement of the user's limbs has a single pattern within a single gait cycle. In contrast, sensor data representing the movement of the user's torso has two patterns within a single gait cycle. The estimation accuracy of the ground reaction force in the cases C2 to C8 being better than that in the case C1 is presumed to be because sensor data having different patterns within a single gait cycle are used.

As illustrated in FIG. 10, in the case C9, the mean squared error of the normalized ground reaction force on the left foot was around 0.112 [(m/s²)²]. In the case C10, the mean squared error of the normalized ground reaction force on the left foot was around 0.066 [(m/s²)²]. In the cases C9 and C10, sensor data representing the movement of the user's feet and sensor data representing the movement of the user's thighs are respectively used. The user's feet and the user's thighs are the body parts closest to the walking surface among the user's body parts. In the cases C9 and C10, the ground reaction force is presumed to be estimated with some degree of estimation accuracy because the sensor data representing the movement of body parts close to the walking surface are used.

As illustrated in FIG. 10, in the case C11, the mean squared error of the normalized ground reaction force on the left foot was around 0.068 [(m/s²)²]. In the case C11, only sensor data representing the movement of the user's waist is used. The estimation results of the case C11 demonstrate that the ground reaction force can be estimated with some degree of accuracy even when only sensor data representing the movement of the user's waist is used. The reason for this is presumably that the movement of the user in the up-down direction while walking is reflected in the movement of the torso including the waist.

As illustrated in FIG. 10, the mean squared errors in the cases C12 and C13 were smaller than the mean squared error in the case C11. In other words, the estimation accuracy of the ground reaction force in the cases C12 to C13 was improved compared to that in the case C11. In the cases C12 and C13, sensor data representing the movement of at least any of the user's wrists and ankles is used in addition to the sensor data representing the movement of the user's waist, as described above. In other words, in the cases C12 and C13, sensor data representing the movement of the user's limbs, including at least any of the user's wrists and ankles, are used in addition to sensor data representing the movement of the user's torso including the user's waist. As described above, sensor data representing the movement of the user's limbs and sensor data representing the movement of the user's torso have significantly different patterns from each other within a single gait cycle. The estimation accuracy of the ground reaction force in the cases C12 and C13 is presumed to be better than that in the case C11 because sensor data having different patterns within a single gait cycle are used.

As illustrated in FIG. 10, the mean squared error of the normalized ground reaction force on the left foot in the case C6 and the mean squared error of the normalized ground reaction force on left foot in the case C13 were both around 0.04 [(m/s²)²]. The mean squared errors for the cases C6 and C13 were the smallest among the cases C1 through C13. In other words, the estimation accuracy of the ground reaction force in the cases C6 and C13 was the highest among the cases C1 through C13.

Next, comparison results of comparing measured values and estimated values of the ground reaction force of subjects will be described. First, the subjects used in the comparison results will be described while referring to FIG. 11.

FIG. 11 illustrates an example of subjects. The subjects have diverse physical characteristics.

A subject SU1 is male, 33 years of age, has a height of 171 [cm], and a weight of 100 [kg]. Physical characteristics of subject SU1 are that he is a heavy weight male.

A subject SU2 is female, 70 years of age, has a height of 151 [cm], and a weight of 39 [kg]. Physical characteristics of the subject SU2 are that she is a light weight female.

A subject SU3 is female, 38 years of age, has a height of 155 [cm], and a weight of 41 [kg]. Physical characteristics of the subject SU3 are that she is a light weight young female.

A subject SU4 is female, 65 years of age, has a height of 149 [cm], and a weight of 70 [kg]. Physical characteristics of the subject SU4 are that she is a heavy weight female.

A subject SU5 is male, 22 years of age, has a height of 163 [cm], and a weight of 65 [kg]. Physical characteristics of subject SU5 are that he is a male of average height and average weight.

A subject SU6 is female, 66 years of age, has a height of 149 [cm], and a weight of 47 [kg]. Physical characteristics of the subject SU6 are that she is a short female.

A subject SU7 is female, 65 years of age, has a height of 148 [cm], and a weight of 47 [kg]. Physical characteristic of the subject SU7 are that she is a short female.

A subject SU8 is male, 57 years of age, has a height of 178 [cm], and a weight of 81 [kg]. Physical characteristics of the subject SU8 are that he is a tall male.

### Comparative Example 1

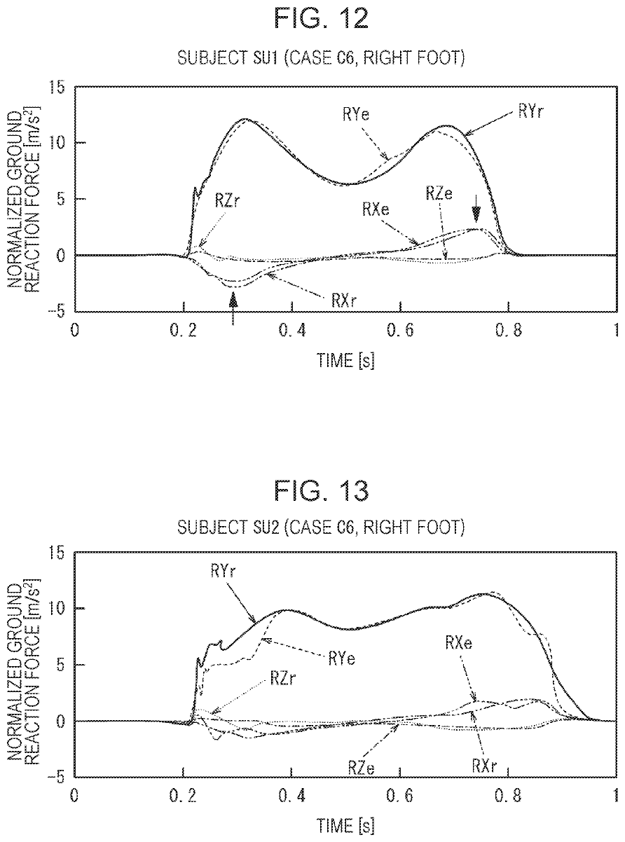

FIGS. 12 to 19 illustrate graphs of an example of measured values and estimated values of the ground reaction force of subjects. In FIGS. 12 to 19, a transformer generated using the sensor data in the case C6 was used. The horizontal and vertical axes in FIGS. 12 to 19 are the same as the horizontal and vertical axes in FIG. 5, respectively.

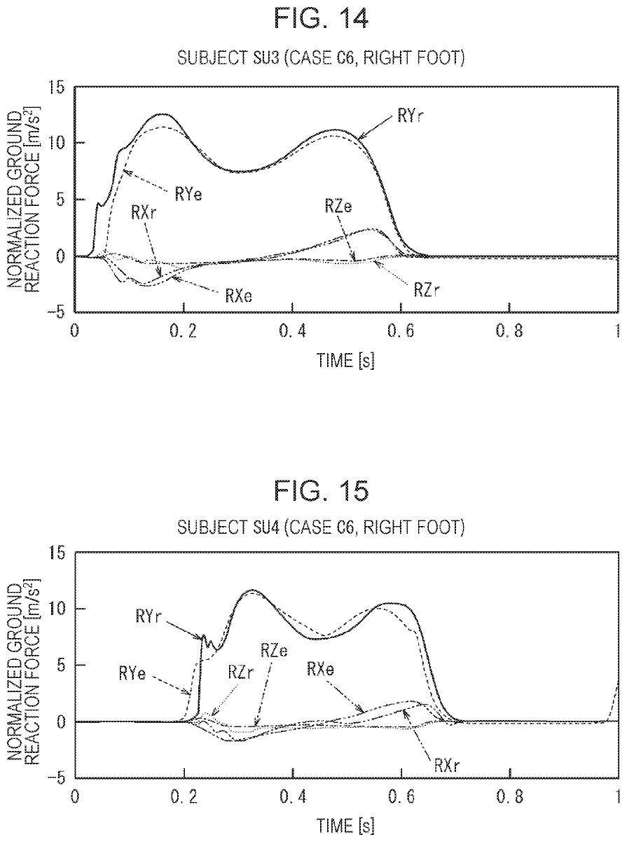

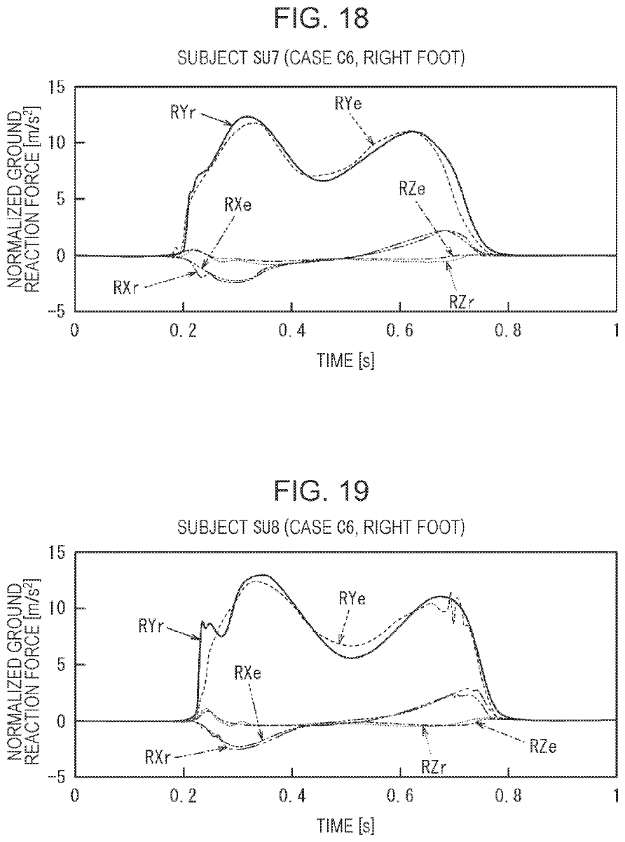

FIGS. 12 to 15, FIG. 18, and FIG. 19 illustrate graphs of measured values and estimated values of the ground reaction force on the right foot. FIG. 12 is a graph for the subject SU1. FIG. 13 is a graph for the subject SU2. FIG. 14 is a graph for the subject SU3. FIG. 15 is a graph for the subject SU4. FIG. 18 is a graph for the subject SU7. FIG. 19 is a graph for the subject SU8.

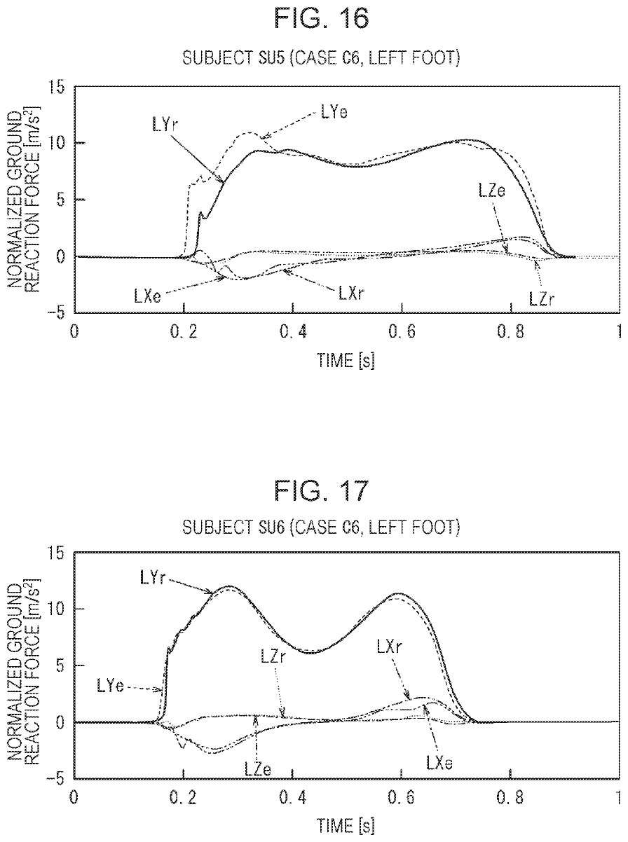

FIGS. 16 and 17 illustrate graphs of measured values and estimated values of the ground reaction force on the left foot. FIG. 16 is a graph for the subject SU5. FIG. 17 is a graph for the subject SU6.

In the following drawings, normalized ground reaction forces RXr, RYr, and RZr are measured values of the ground reaction force applied to the subject's right foot. Normalized ground reaction forces RXe, RYe, and RZe are estimated values of the ground reaction force applied to the subject's right foot.

The normalized ground reaction forces RXr and RXe are the normalized ground reaction force along the X-axis of the global coordinate system out of the normalized ground reaction force acting on the subject's right foot. The direction from in front of to behind the user is taken to be the positive direction of the normalized ground reaction forces RXr and RXe.

The normalized ground reaction forces RYr and RYe are the normalized ground reaction force along the Y-axis of the global coordinate system out of the normalized ground reaction force acting on the subject's right foot. The direction from below to above the user is taken to be the positive direction of the normalized ground reaction forces RYr and RYe.

The normalized ground reaction forces RZr and RZe are the normalized ground reaction force along the Z-axis of the global coordinate system out of the normalized ground reaction force acting on the subject's right foot. The direction from the left to the right of the user is taken to be the positive direction of the normalized ground reaction forces RZr and RZe.

Normalized ground reaction forces LXr, LYr, and LZr are measured values of the ground reaction force applied to the subject's left foot. Normalized ground reaction forces LXe, LYe, and LZe are estimated values of the ground reaction force applied to the subject's left foot.

The normalized ground reaction forces LXr and LXe are the normalized ground reaction force along the X-axis of the global coordinate system out of the normalized ground reaction force acting on the subject's left foot. The direction from in front of to behind the user is taken to be the positive direction of the normalized ground reaction forces LXr and LXe.

The normalized ground reaction forces LYr and LYe are the normalized ground reaction force along the Y-axis of the global coordinate system out of the normalized ground reaction force acting on the subject's left foot. The direction from below to above the user is taken to be the positive direction of the normalized ground reaction forces LYr and LYe.

The normalized ground reaction forces LZr and LZe are the normalized ground reaction force along the Z-axis of the global coordinate system out of the normalized ground reaction force acting on the subject's left foot. The direction from the left to the right of the user is taken to be the positive direction of the normalized ground reaction forces LZr and LZe.

As illustrated in FIGS. 12 to 19, the estimated values of the normalized ground reaction force agreed relatively well with the measured values of the normalized ground reaction force. The subjects SU1 to SU8 have diverse physical characteristics, as described above with reference to FIG. 11. These results demonstrate that the ground reaction force can be estimated with good accuracy regardless of physical characteristics when a transformer is generated using the sensor data of the case C6.

### Comparative Example 2

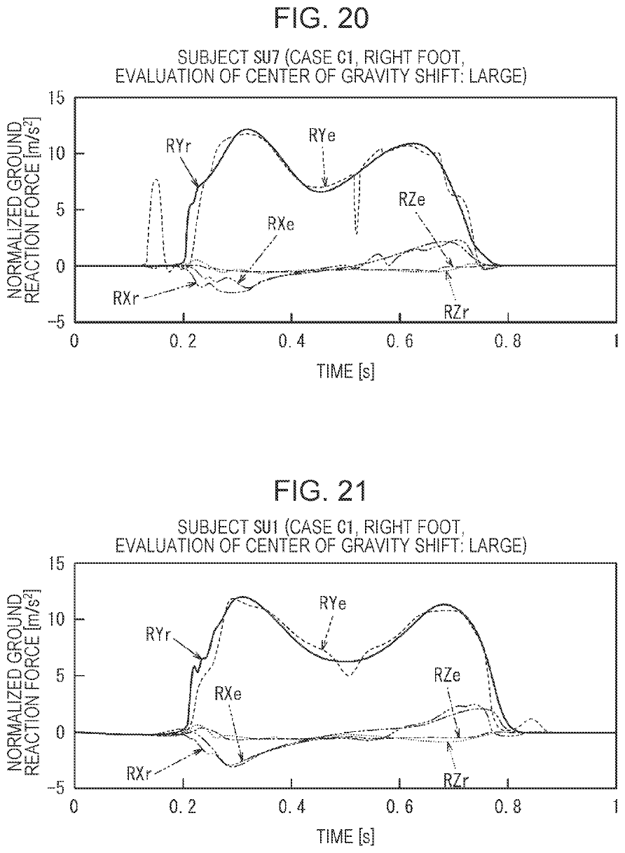

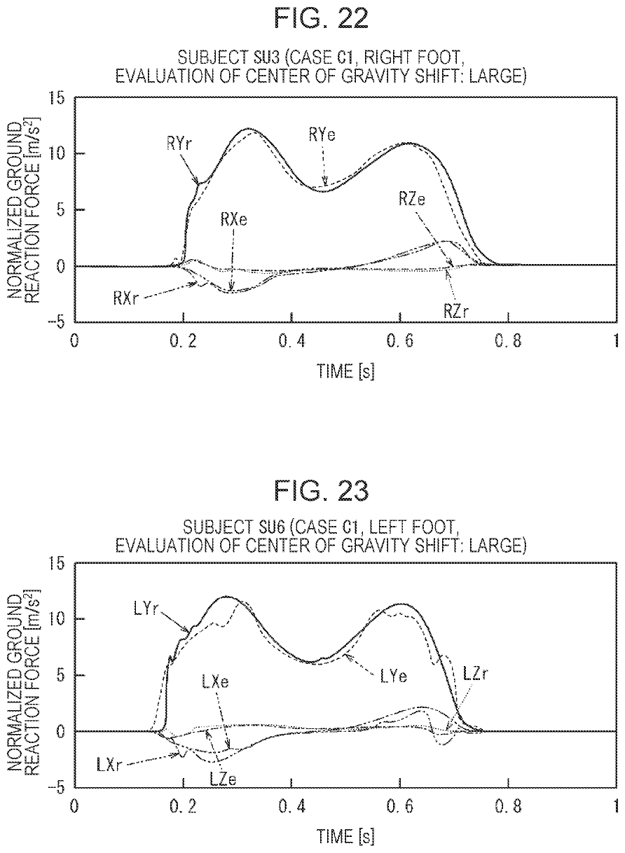

FIGS. 20 to 23 illustrate graphs of an example of measured values and estimated values of the ground reaction force of subjects evaluated as having a large center of gravity shift. FIG. 20 is a graph of the ground reaction force applied to the right foot of the subject SU7. FIG. 21 is a graph of the ground reaction force applied to the right foot of the subject SU1. FIG. 22 is a graph of the ground reaction force applied to the right foot of the subject SU3. FIG. 23 is a graph of the ground reaction force applied to the left foot of the subject SU6.

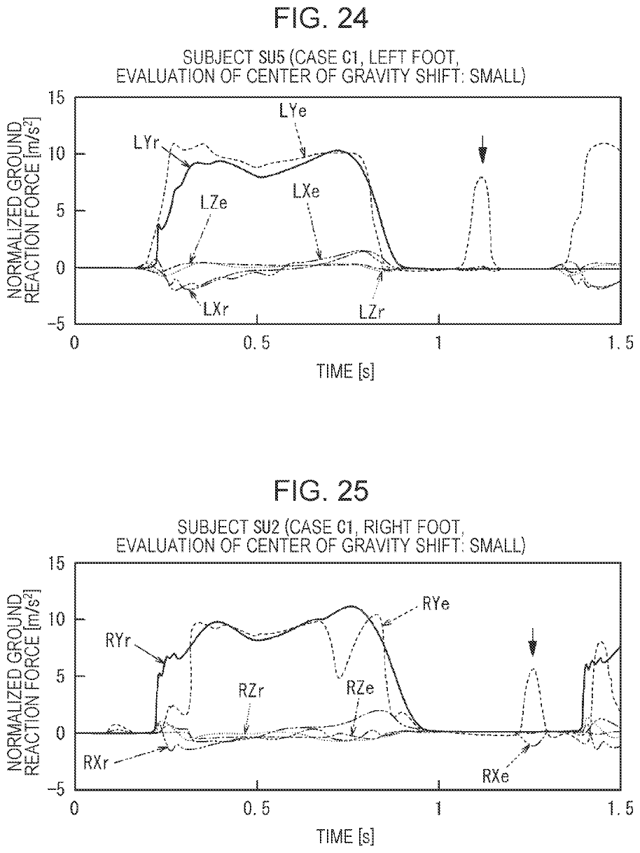

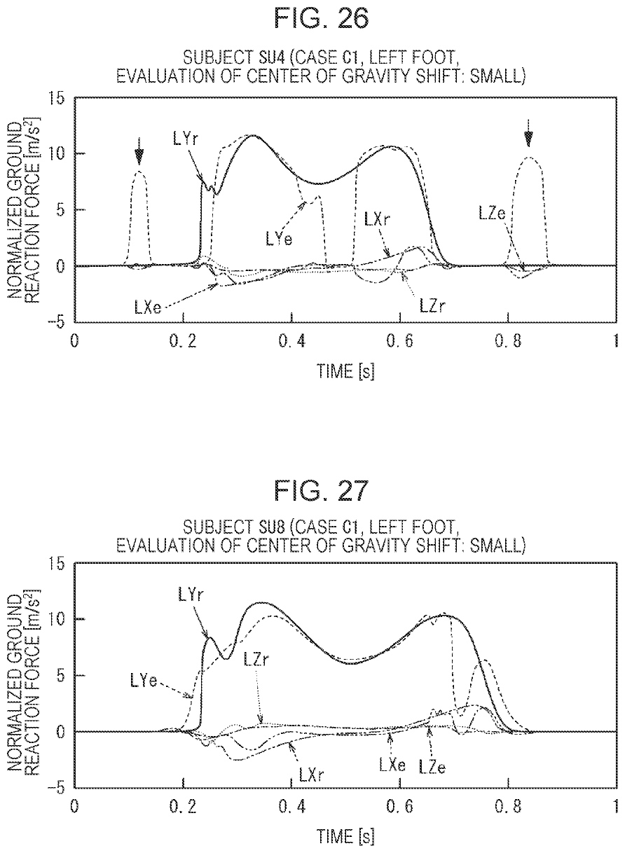

FIGS. 24 to 27 illustrate graphs of an example of measured values and estimated values of the ground reaction force of subjects evaluated as having a small center of gravity shift. FIG. 24 is a graph of the ground reaction force applied to the left foot of the subject SU5. FIG. 25 is a graph of the ground reaction force applied to the right foot of the subject SU2. FIG. 26 is a graph of the ground reaction force applied to the left foot of the subject SU4. FIG. 27 is a graph of the ground reaction force applied to the left foot of the subject SU8.

In FIGS. 20 to 27, a transformer generated using the sensor data in the case C1 was used. The horizontal and vertical axes in FIGS. 20 to 23 are the same as the horizontal and vertical axes in FIG. 5, respectively.

A subject evaluated as having a large center of gravity shift means a subject for which the shift of the center of gravity of the subject in the up-down direction is large. As illustrated in FIGS. 20 to 23, the normalized ground reaction forces RYr, RYe, LYr, and LYe along the Y-axis each have two maxima and a minimum positioned between the two maxima. The minimum is caused by the user's center of gravity shifting upward relative to the walking surface during the midstance. The larger the shift of the user's center of gravity in the up-down direction, the larger the difference between the two maxima and the minimum positioned between the two maxima in each of the normalized ground reaction forces RYr, RYe, LYr, and LYe along the Y-axis, as illustrated in FIGS. 20 to 23.

A subject evaluated as having a small center of gravity shift means a subject for which the shift of the center of gravity of the subject in the up-down direction is small. The larger the shift of the user's center of gravity in the up-down direction, the smaller the difference between the two maxima and the minimum positioned between the two maxima in each of the normalized ground reaction forces RYr, RYe, LYr, and LYe along the Y-axis, as illustrated in FIGS. 24 to 27.

For subjects evaluated as having a large center of gravity shift, the estimated values of the normalized ground reaction force agreed relatively well with the measured values of the normalized ground reaction force, as illustrated in FIGS. 20 to 23. In the case C1, only sensor data representing the movement of the user's head is used. For subjects evaluated as having a large center of gravity shift, the center of gravity shift of the subject in the up-down direction is greater and the movement of the subject in the up-down direction is larger. For subjects evaluated as having a large center of gravity shift, the characteristics of the subject's movement while walking can be reflected in the movement of the user's head due to the greater amount of movement of the subject in the up-down direction. Therefore, for subjects evaluated as having a large center of gravity shift, the estimated values of the normalized ground reaction force are presumed to be in relatively good agreement with the measured values of the normalized ground reaction force.

For subjects evaluated as having a small center of gravity shift, when compared to FIGS. 20 to 23, the estimated values of the normalized ground reaction force did not agree particularly well with the measured values of the normalized ground reaction force, as illustrated in FIGS. 24 to 27. For subjects evaluated as having a small center of gravity shift, the shift of the center of gravity of the subject in the up-down direction is smaller and the amount of movement of the subject in the up-down direction is also smaller. For subjects evaluated as having a small center of gravity shift, the characteristics of the subject's movement while walking are less likely to be reflected in the movement of the user's head compared to subjects evaluated as having a large center of gravity shift, because the amount of movement of the subject in the up-down direction is smaller. Therefore, for subjects evaluated as having a small center of gravity shift, the estimated values of the normalized ground reaction force are presumed to not agree particularly well with the measured values of the normalized ground reaction force when compared to FIGS. 20 to 23.

Thus, for subjects evaluated as having a small center of gravity shift, the estimation accuracy of the ground reaction force using the sensor data for the case C1 is lower than for subjects evaluated as having a large center of gravity shift. In contrast, for data of subjects evaluated as having a large center of gravity shift, the ground reaction force can be estimated relatively accurately even when the sensor data for the case C1 is used.

### Comparative Example 3

Estimated values of the ground reaction force for when combinations of different sensor data are used for the same subject will be described.

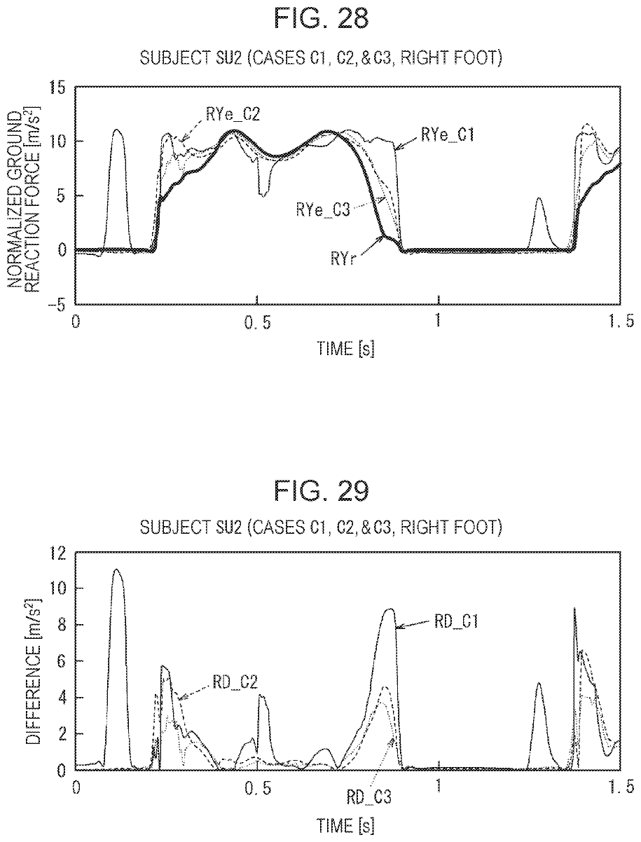

FIGS. 28 to 35 illustrate graphs of the ground reaction force applied to the right foot of the subject SU2.

FIG. 28 is a graph of the estimated values of the ground reaction force when the sensor data of the cases C1, C2, and C3 are used. FIG. 29 is a graph illustrating the differences between the measured values and the estimated values of the ground reaction force illustrated in FIG. 28. The horizontal and vertical axes in FIG. 28 are the same as the horizontal and vertical axes in FIG. 5, respectively. The horizontal axis in FIG. 29 represents time[s]. The vertical axis in FIG. 29 represents difference [m/s²].

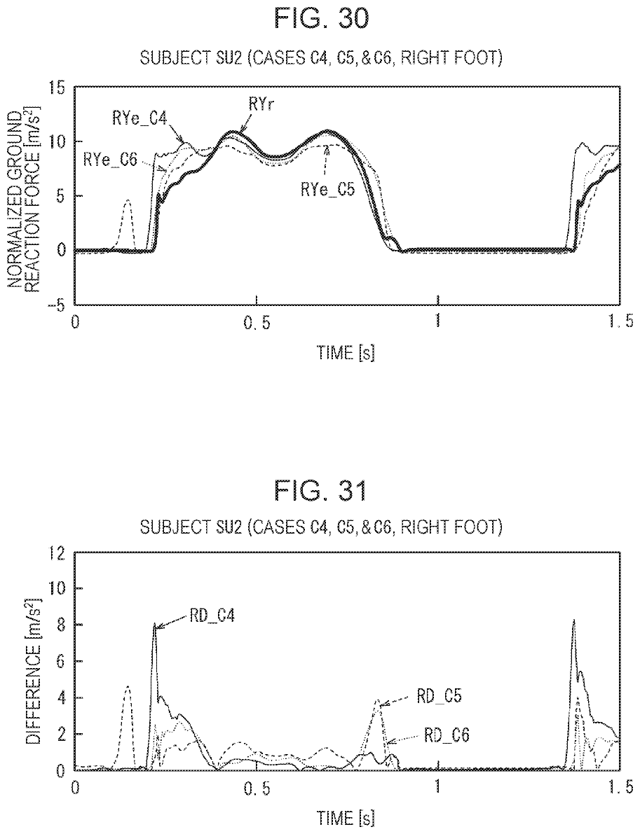

FIG. 30 is a graph of the estimated values of the ground reaction force when the sensor data of the cases C4, C5, and C6 are used. FIG. 31 is a graph illustrating the differences between the measured values and the estimated values of the ground reaction force illustrated in FIG. 30. The horizontal and vertical axes in FIG. 30 are the same as the horizontal and vertical axes in FIG. 5, respectively. The horizontal and vertical axes in FIG. 31 are the same as the horizontal and vertical axes in FIG. 29, respectively.

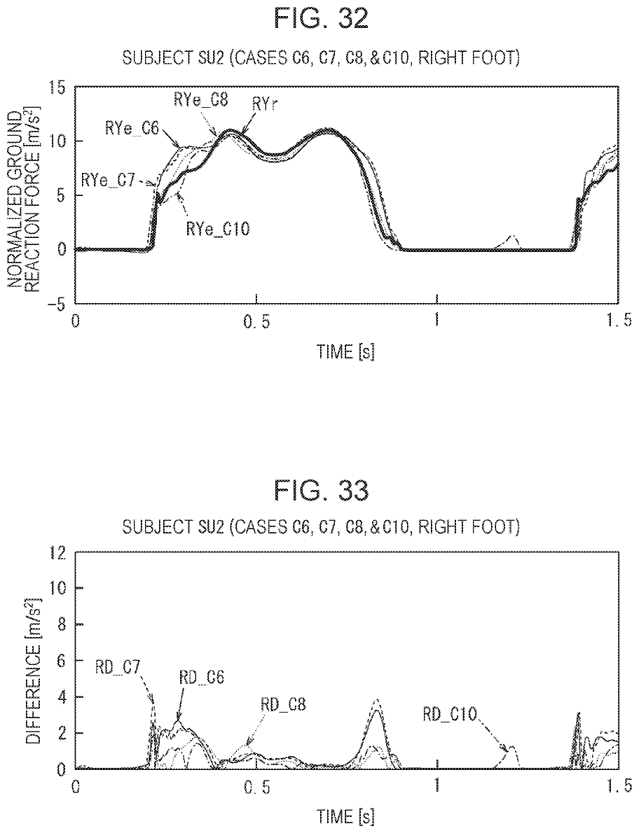

FIG. 32 is a graph of the estimated values of the ground reaction force when the sensor data of the cases C6, C7, C8, and C10 are used. FIG. 33 is a graph illustrating the differences between the measured values and the estimated values of the ground reaction force illustrated in FIG. 32. The horizontal and vertical axes in FIG. 32 are the same as the horizontal and vertical axes in FIG. 5, respectively. The horizontal and vertical axes in FIG. 33 are the same as the horizontal and vertical axes in FIG. 29, respectively.

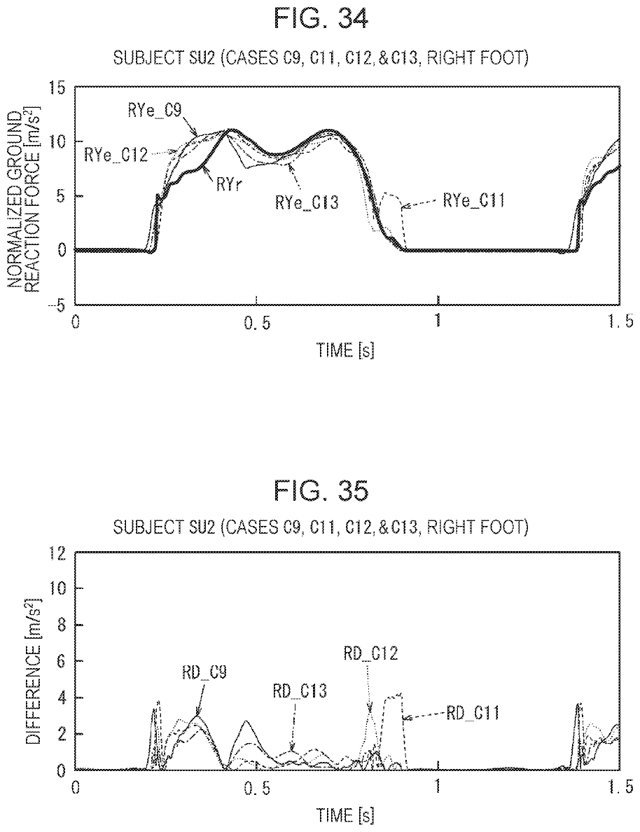

FIG. 34 is a graph of the estimated values of the ground reaction force when the sensor data of the cases C9, C11, C12, and C13 are used. FIG. 35 is a graph illustrating the differences between the measured values and the estimated values of the ground reaction force illustrated in FIG. 34. The horizontal and vertical axes in FIG. 34 are the same as the horizontal and vertical axes in FIG. 5, respectively. The horizontal and vertical axes in FIG. 35 are the same as the horizontal and vertical axes in FIG. 29, respectively.

In FIGS. 32 to 35, normalized ground reaction forces RYe_C1 to RYe_C13 are normalized ground reaction forces RYe for when the sensor data of the cases C1 to C13 are used, respectively. Differences RD_C1 to RD_C13 are the differences between the normalized ground reaction forces RYe_C1 to RYe_C13 and the normalized ground reaction force RYr, respectively.

For the subject SU2, the difference between the measured values and the estimated values of the ground reaction force was smallest when sensor data from the cases C6 and C13 were used, among the cases C1 to C13. The subject SU2 is a subject evaluated as having a small center of gravity shift. Even for subjects evaluated as having a small center of gravity shift, we can see that the ground reaction force can be estimated with good accuracy using the sensor data for the case C6 and the case C13.

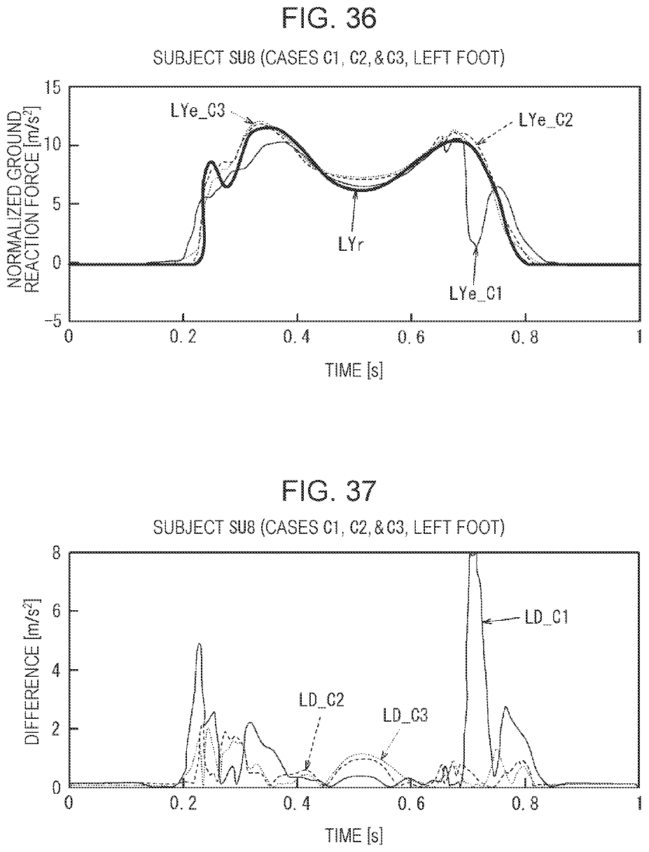

FIGS. 36 to 43 illustrate graphs of the ground reaction force applied to the left foot of the subject SU8.

FIG. 36 is a graph of the estimated values of the ground reaction force when the sensor data of the cases C1, C2, and C3 are used. FIG. 37 is a graph illustrating the differences between the measured values and the estimated values of the ground reaction force illustrated in FIG. 36. The horizontal and vertical axes in FIG. 36 are the same as the horizontal and vertical axes in FIG. 5, respectively. The horizontal and vertical axes in FIG. 37 are the same as the horizontal and vertical axes in FIG. 29, respectively.

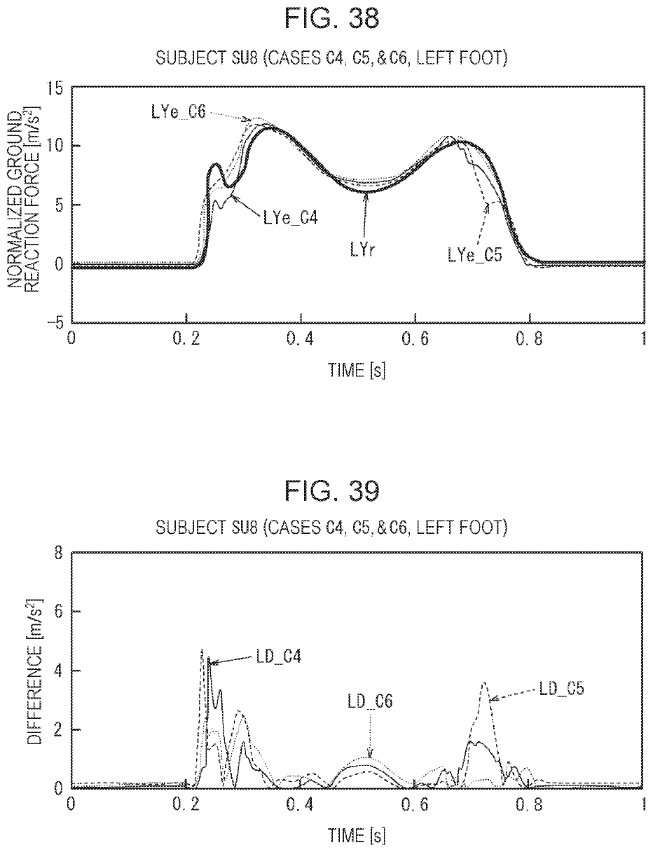

FIG. 38 is a graph of the estimated values of the ground reaction force when the sensor data of the cases C4, C5, and C6 are used. FIG. 39 is a graph illustrating the differences between the measured values and the estimated values of the ground reaction force illustrated in FIG. 38. The horizontal and vertical axes in FIG. 38 are the same as the horizontal and vertical axes in FIG. 5, respectively. The horizontal and vertical axes in FIG. 39 are the same as the horizontal and vertical axes in FIG. 29, respectively.

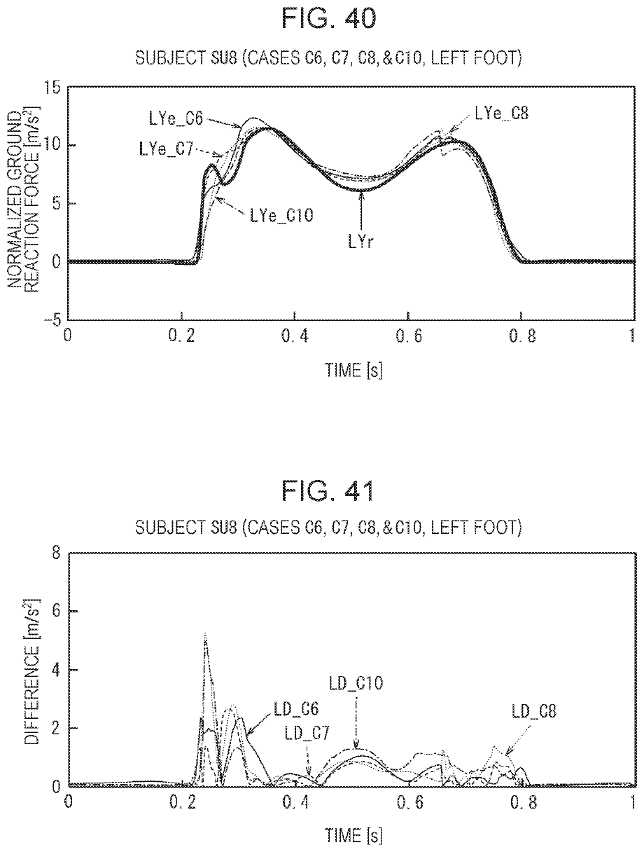

FIG. 40 is a graph of the estimated values of the ground reaction force when the sensor data of the cases C6, C7, C8, and C10 are used. FIG. 41 is a graph illustrating the differences between the measured values and the estimated values of the ground reaction force illustrated in FIG. 40. The horizontal and vertical axes in FIG. 40 are the same as the horizontal and vertical axes in FIG. 5, respectively. The horizontal and vertical axes in FIG. 41 are the same as the horizontal and vertical axes in FIG. 29, respectively.

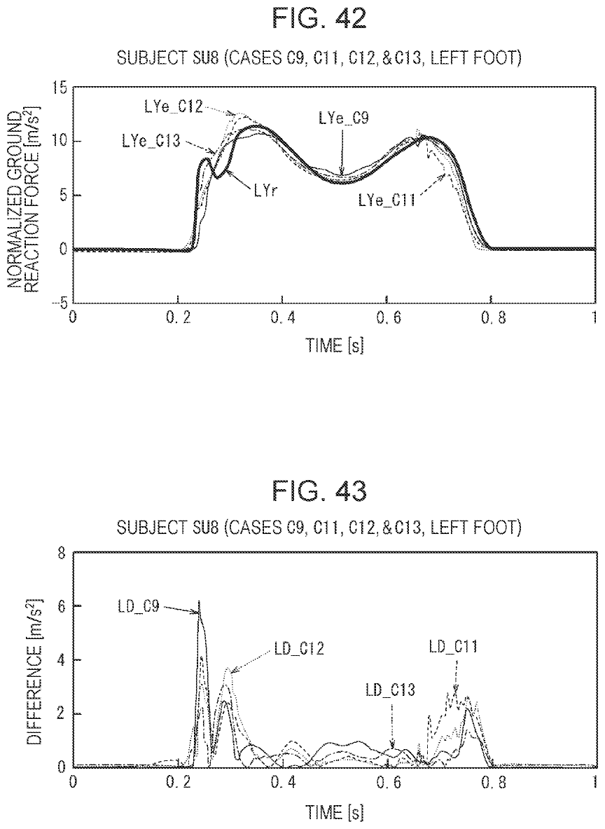

FIG. 42 is a graph of the estimated values of the ground reaction force when the sensor data of the cases C9, C11, C12, and C13 are used. FIG. 43 is a graph illustrating the differences between the measured values and the estimated values of the ground reaction force illustrated in FIG. 42. The horizontal and vertical axes in FIG. 42 are the same as the horizontal and vertical axes in FIG. 5, respectively. The horizontal and vertical axes in FIG. 43 are the same as the horizontal and vertical axes in FIG. 29, respectively.

In FIGS. 36 to 43, normalized ground reaction forces LYe_C1 to LYe_C13 are normalized ground reaction forces LYe for when the sensor data of the cases C1 to C13 are used, respectively. Differences LD_C1 to LD_C13 are the differences between the normalized ground reaction forces LYe_C1 to LYe_C13 and the normalized ground reaction force LYr, respectively.

For the subject SU8, the difference between the measured values and the estimated values of the ground reaction force was smallest when sensor data from the cases C6 and C13 were used, among the cases C1 to C13. The subject SU8 is a subject evaluated as having a small center of gravity shift. Even for subjects evaluated as having a small center of gravity shift, we can see that the ground reaction force can be estimated with good accuracy using the sensor data for the case C6 and the case C13.

### [Processing For Acquiring Information About Gait]

The controller 27 may acquire any information about a user's gait based on estimated values of the ground reaction force.

As an example, the information about the user's gait may be the landing timing and the gait cycle of the user. The controller 27 may acquire at least any one of the landing timing and the gait cycle of the user based on estimated values of the ground reaction force. For example, in FIG. 5, the timing of the rise of the normalized ground reaction force LY, indicated by an arrow, corresponds to the landing timing of the left foot of the user. In addition, the timing of the rise of the normalized ground reaction force RY, indicated by an arrow, corresponds to the landing timing of the right foot of the user. The controller 27 acquires the landing timing of the user by identifying the timing of the rise of the estimated values of the ground reaction force. The controller 27 identifies and acquires the user's gait cycle based on the acquired landing timing of the user.

As another example, information about the user's gait may be information on the user's stride length. The controller 27 may acquire the information on the user's stride length based on the estimated values of the ground reaction force. For example, the controller 27 acquires the landing timing of the user's left foot and the landing timing of the user's right foot based on the estimated values of the ground reaction force as described above. The controller 27 calculates and acquires the stride length of the user based on the landing timing of the user's left foot, the landing timing of the user's right foot, and the velocity of the user with respect to the X-axis of the global coordinate system. The controller 27 may acquire information on the velocity of the user along the X-axis of the global coordinate system from the sensor device 10.

As yet another example, information about the user's gait may be at least any of information about the load on the user's joints while walking and information about the load on the user's muscles while walking. In this case, the controller 27 may acquire at least any of the information on the load on the user's joints and the load on the user's muscles while walking by performing an inverse dynamics analysis on the estimated values of the ground reaction force.

The controller 27 may cause the notification unit 23 to report the information about the user's gait acquired by the controller 27. For example, the controller 27 may display the information about the user's gait on the display of the output unit 24, or may output the information about the user's gait as audio to the speaker of output unit 24.

### [Evaluation Determination Processing]

The controller 27 may evaluate the user's gait based on estimated values of the ground reaction force.

As an example, an evaluation of the user's gait may be an evaluation of whether or not the user's heel strike during the loading response described above with reference to FIG. 3 is adequate. The controller 27 may evaluate whether the user's heel strike during the loading response is adequate based on the estimated values of the ground reaction force. For example, in FIG. 5, the normalized ground reaction force LY has two maxima, as indicated by the arrows. The first of the two maxima corresponds to the ground reaction force during the loading response. The loading response is the period during which a heel strike from the heel of the foot is observed, and therefore whether or not the user's heel strike during the loading response is adequate can be evaluated based on the first maximum. The controller 27 evaluates whether the user's heel strike during the loading response is adequate by analyzing the first of two maxima of the normalized ground reaction or ground reaction force along the Y-axis.

As another example, the evaluation of the user's gait may be an evaluation of whether the user's push off, as described above with reference to FIG. 3, is adequate. The controller 27 may evaluate whether the user's push off is adequate based on the estimated values of the ground reaction force. For example, in FIG. 5, the second of the two maxima of the normalized ground reaction force LY indicated the arrows corresponds to the ground reaction force at the push off timing of the user. The controller 27 evaluates whether or not the user's push off is adequate by analyzing the second of the two maxima of the normalized ground reaction force or ground reaction force along the Y-axis.

As yet another example, the evaluation of the user's gait may be an evaluation of the shift of the user's center of gravity in the up-down direction. The controller 27 may evaluate the shift of the user's center of gravity in the up-down direction based on the estimated values of the ground reaction force. For example, in FIG. 5, the normalized ground reaction force LY has two maxima and one minimum positioned between the two maxima as indicated by the arrows. As described above, the larger the shift of the user's center of gravity in the up-down direction, the larger the difference between the two maxima and the minimum positioned between the two maxima. The controller 27 may determine whether the shift of user's center of gravity in the up-down direction is evaluated as large based on the difference between the two maxima and the minimum positioned between the two maxima in the normalized ground reaction force or the ground reaction force along the y-axis.

As an evaluation of the shift of the user's center of gravity in the up-down direction, the controller 27 may also determine whether the shift of the user's center of gravity in the up-down direction is evaluated as small. For example, as described above, during the swing phase, the foot is not in contact with the walking surface, and therefore no ground reaction force is applied to the foot. However, for subjects evaluated as having a small center of gravity shift, when the ground reaction force is estimated using the sensor data in the case C1, the normalized ground reaction forces LYe and RYe might not be zero during the swing phase, for example, as indicated in FIGS. 24, 25, and 26 by the arrows. Therefore, the controller 27 may estimate the normalized ground reaction force or the ground reaction force along the Y-axis using the sensor data in the case C1. Furthermore, if the estimated normalized ground reaction force or ground reaction force is not zero in the swing phase, the controller 27 may determine that the shift of the user's center of gravity is evaluated as smaller than the center of gravity shift would be if the ground reaction force were zero in the swing phase. The controller 27 may estimate the normalized ground reaction force or ground reaction force along the Y-axis using the sensor data in the case C1 to evaluate the shift of the user's center of gravity, even if the sensor devices 10 that transmitted the sensor data to the electronic device 20 include a sensor device other than the sensor device 10A.

As yet another example, the evaluation of the user's gait may be an evaluation of whether the user's foot is acting as a brake or an accelerator. Based on the estimated values of the ground reaction force, the controller 27 may evaluate whether the user's foot is acting as a brake or an accelerator. For example, in FIG. 12, the normalized ground reaction force RXe has a negative peak value and a positive peak value as indicated by the arrows. The negative peak value is caused by the user's foot landing on the walking surface and acting as a brake. The positive peak value is caused by the user's foot pushing off the walking surface and acting as an accelerator. By analyzing the positive and negative peak values of the normalized ground reaction force or ground reaction force along the X-axis, the controller 27 evaluates whether the user's foot is functioning as a brake or an accelerator.

As yet another example, the evaluation of the user's gait may be an evaluation of whether the user's stride length is reasonable for his or her height. For example, the controller 27 acquires information on the user's stride length based on the estimated values of the ground reaction force, as described above. The controller 27 acquires information on the user's height from the storage unit 26 and compares the user's height and the stride length in order to evaluate whether the user's stride length is reasonable for his or her height.

The controller 27 may inform the user of the determined evaluation using the notification unit 23. For example, the controller 27 may display information on the determined evaluation on the display of the output unit 24, or may output information on the determined evaluation as audio to the speaker of the output unit 24. Alternatively, the controller 27 may cause the vibration unit 25 to vibrate with a vibration pattern in accordance with the determined evaluation.

### [Processing for Transmission to External Device]

The controller 27 may generate a measurement signal representing at least any of an estimated value of the ground reaction force, acquired information about gait, and a determined evaluation. The controller 27 may transmit the generated measurement signal to the communication unit 21 to any external device.

The controller 27 may transmit the measurement signal to any sensor device 10 including the notification unit 13 as an external device by using the communication unit 21. In this case, in the sensor device 10, the controller 16 receives the measurement signal via the communication unit 11. The controller 16 causes the notification unit 13 to report the information represented by the measurement signal. For example, the controller 16 causes the output unit 14 to output the information represented by the measurement signal. This configuration allows the user to ascertain, for example, the ground reaction force.

The controller 27 may transmit the measurement signal using the communication unit 21 to an earphone as an external device, if the sensor device 10A is an earphone or is contained in an earphone, for example. In this case, in the sensor device 10A, the controller 16 receives the measurement signal via the communication unit 11. In the sensor device 10A, the controller 16 causes the notification unit 13 to report the information represented by the measurement signal. For example, in the sensor device 10A, the controller 16 causes the speaker of the output unit 14 to output the information represented in the measurement signal as audio. This configuration allows the user to be informed of information regarding the ground reaction force or the like via audio. Informing the user via audio reduces the likelihood of the user's walk being disturbed.

### (System Operation)

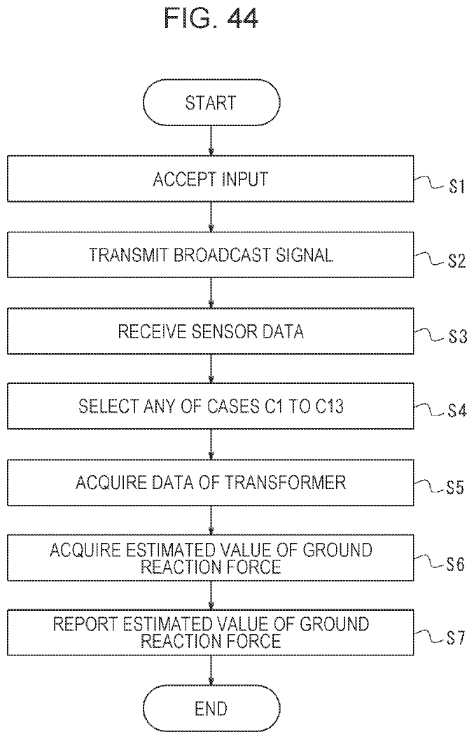

FIG. 44 is a flowchart illustrating operation of ground reaction force estimation processing performed by the electronic device 20 illustrated in FIG. 1. This operation corresponds to an example of an information processing method according to this embodiment. Upon accepting an input instructing execution of ground reaction force estimation processing via the input unit 22, the controller 27 starts processing of Step S1.

The controller 27 accepts an input instructing execution of the ground reaction force estimation processing via the input unit 22 (Step S1). This input is input from the input unit 22 by the user wearing the sensor device 10.

The controller 27 transmits a signal instructing the start of data detection to multiple sensor devices 10 as a broadcast signal via the communication unit 21 (Step S2). After the processing of Step S2 is performed, sensor data is transmitted from at least one sensor device 10 to the electronic device 20.

The controller 27 receives the sensor data from the at least one sensor device 10 via the communication unit 21 (Step S3).

The controller 27 selects any of the cases C1 to C13 in accordance with the type of sensor device 10 that transmitted the sensor data to the electronic device 20 (Step S4). The controller 27 acquires the data of the transformer 30 used in the cases C1 to C13 selected in the processing of Step S4 from the storage unit 26 (Step S5).

The controller 27 inputs the sensor data for the cases C1 to C13 selected in the processing of Step S4 to the transformer whose data was acquired in the processing of Step S5, and acquires estimated values of the ground reaction force from the transformer (Step S6).

The controller 27 reports the estimated values of the ground reaction force acquired in the processing of Step S6 via the notification unit 23 (Step S7).

After executing the processing of Step S7, the controller 27 terminates the estimation processing. After terminating the estimation processing, the controller 27 may perform the estimation processing again when the user walks a set number of steps. This set number of steps may be input in advance by the user from the input unit 22. In the estimation processing to be performed again, the controller 27 may start from the processing of Step S3. The controller 27 may repeat the estimation processing every set number of steps taken by the user until an input instructing termination of the estimation processing is received from the input unit 22. An input instructing termination of the estimation processing is input by the user, for example, through the input unit 22. When the user finishes walking, for example, he or she inputs an input from the input unit 22 instructing termination of the estimation processing. When the controller 27 receives an input instructing termination of the estimation processing, the controller 27 may transmit a signal instructing termination of data detection as a broadcast signal to the multiple sensor devices 10 via the communication unit 21. In the sensor device 10, the controller 16 may terminate data detection when a signal instructing termination of data detection is received by the communication unit 11.

Thus, in the electronic device 20 serving as an information processing device, the controller 27 acquires estimated values of the ground reaction force applied to the user by using sensor data and a trained model. In this embodiment, by using the trained model, estimated values of the ground reaction force applied to the user can be acquired without the use of a large-scale device such as a ground reaction force meter. With this configuration, in this embodiment, estimated values of the ground reaction force applied to the user can be acquired with a simpler configuration. Thus, in this embodiment, an improved technology for measuring (estimating) the ground reaction force is provided.

Furthermore, in this embodiment, the controller 27 may acquire sensor data from at least one sensor device 10 worn on a body part of the user. Advantages of using data detected by the sensor device 10 as sensor data will be described below with comparison to Comparative Examples 1 and 2.

As Comparative Example 1, a case in which the ground reaction force is measured using a ground reaction force meter is considered. In this case, the ground reaction force cannot be measured unless the user walks on the area where the ground reaction force meter is installed. In most cases, the ground reaction force meter is installed indoors such as in a special laboratory. Therefore, in Comparative Example 1, the user cannot relax and walk as he or she normally would because the user needs to walk in a special laboratory or the like where the ground reaction force meter is installed. If the user cannot walk as he or she normally would, the ground reaction force cannot be measured correctly. In addition, due to the finite size of the ground reaction force meter, the ground reaction force meter can only measure the ground reaction force for a limited number of steps. Therefore, in Comparative Example 1, measuring the ground reaction force while the user walks outdoors for a long period of time is difficult.

In contrast to Comparative Example 1, when data detected by the sensor device 10 is used as sensor data, the estimated values of the ground reaction force can be acquired wherever the user walks, as long as the user is wearing the sensor device 10. Even if the user walks for an extended period of time, the estimated values of the ground reaction force can be acquired as long as the user is wearing the sensor device 10. When data detected by the sensor device 10 is used as sensor data in this way, the estimated values of the ground reaction force can be acquired regardless of the location where the user walks and the time at which the user walks. With this configuration, the information processing system 1 can be used for any application, including rehabilitation.

As Comparative Example 2, a case in which the ground reaction force applied to a user is measured using a shoe equipped with a load sensor built into the sole. When such a shoe having a built-in load sensor is used, the ground reaction force can be measured wherever the user walks as long as the user is wearing the shoe. Even if the user walks for an extended period of time, the ground reaction force can be measured as long as the user is wearing the shoe. However, such load sensors are generally expensive. When shoes having built-in load sensors are used, shoes that match the size of the user's feet need to be prepared.

In contrast to Comparative Example 2, the sensor data detected by the sensor device 10 can be detected by an inertial measurement unit or another device without using a load sensor. Thus, in this embodiment, the estimated values of the ground reaction force of the user can be acquired at lower cost than with a load sensor. When using sensor data detected by the sensor device 10F, for example, if the sensor device 10F is retrofitted to the user's shoe, there is no need to prepare a shoe that matches the size of the user's foot.

Here, in this embodiment, the transformer may be trained to output estimated values of the ground reaction force when input with the sensor data for the case C1. The sensor data for the case C1 is detected by the sensor device 10A. With this configuration, estimated values of the ground reaction force applied to the user can be acquired even when the user wears only the sensor device 10A. In addition, user convenience can be improved because the user only needs to wear the sensor device 10A. Furthermore, if the sensor device 10A is an earphone or is included in an earphone, the user can easily wear sensor device 10A on his or her head. The user being able to easily wear the sensor device 10A on his or her head further improves user convenience. In addition, when only the sensor data detected by sensor device 10A is used, the timings at which multiple sensor devices 10 detect data no longer need to be synchronized. By eliminating the need to synchronize the timings at which the multiple sensor devices 10 detect data, the estimated values of the ground reaction force can be acquired more easily.

In this embodiment, the transformer may be trained to output estimated values of the ground reaction force when input with sensor data from any of the cases C2 to C5. The sensor data in the case C2 is detected by the sensor device 10A and the sensor device 10E-1 or 10E-2, i.e., by two sensor devices 10. The sensor data in the case C3 is detected by the sensor device 10A and the sensor device 10F-1 or 10F-2, i.e., by two sensor devices 10. The sensor data in the case C4 is detected by the sensor device 10A and the sensor device 10D-1 or 10D-2, i.e., by two sensor devices 10. The sensor data in the case C5 is detected by the sensor device 10A and by the sensor device 10B, i.e., by two sensor devices 10. Thus, since the sensor data is detected by two sensor devices 10 in the cases C2 to C5, the user only needs to wear two sensor devices 10. Therefore, user convenience can be improved. As discussed with reference to FIG. 10 above, the estimation accuracy of the ground reaction force was better in the cases C2 to C5 than in the case C1. Therefore, the ground reaction force can be estimated with good accuracy by using the sensor data in the cases C2 to C5.

In this embodiment, the transformer may be trained to output estimated values of the ground reaction force when input with sensor data from any of the cases C6 and C7. The sensor data in the case C6 is detected by the sensor device 10A, the sensor device 10B, and the sensor device 10E-1 or 10E-2, i.e., by three sensor devices 10. The sensor data in the case C7 is detected by the sensor device 10A, the sensor device 10B, and the sensor device 10F-1 or 10F-2, i.e., by three sensor devices 10. Thus, since the sensor data is detected by three sensor devices 10 in the cases C6 and C7, the user only needs to wear three sensor devices 10. Therefore, user convenience can be improved. As discussed with reference to FIG. 10 above, the estimation accuracy of the ground reaction force was better in the cases C6 and C7 than in the case C1. As discussed above with reference to FIG. 10, the estimation accuracy of the ground reaction force in the case C6 was the highest among the cases C1 to C13. Therefore, the ground reaction force can be estimated with good accuracy by using the sensor data in the cases C6 and C7.

### (Another System Configuration)

FIG. 45 is a functional block diagram illustrating the configuration of an information processing system 101 according to another embodiment of the present disclosure.

The information processing system 101 includes the sensor device 10, the electronic device 20, and a server 80. In the information processing system 101, the server 80 functions as an information processing device and acquires estimated values of the ground reaction force applied to the user.

The electronic device 20 and the server 80 can communicate with each other via the network 2. The network 2 may be any network, including mobile object communication networks and the Internet.

The controller 27 of the electronic device 20 receives sensor data from the sensor device 10 via the communication unit 21, in the same or a similar manner to information processing system 1. In the information processing system 101, the controller 27 transmits sensor data to the server 80 via the network 2 by using the communication unit 21.

The server 80 is a server belonging to, for example, a cloud computing system or another computing system. The server 80 includes a communication unit 81, a storage unit 82, and a controller 83.

The communication unit 81 includes at least one communication module that can connect to the network 2. The communication module is, for example, a communication module that is compatible with standards such as wired LAN (Local Area Network) or wireless LAN. The communication unit 81 is connected to the network 2 via wired LAN or wireless LAN using the communication module.

The storage unit 82 includes at least one semiconductor memory, at least one magnetic memory, at least one optical memory, or a combination of at least two of these types of memories. The semiconductor memory is, for example, a RAM or a ROM. The RAM is, for example, an SRAM or a DRAM. The ROM is, for example, an EEPROM. The storage unit 82 may function as a main storage device, an auxiliary storage device, or a cache memory. The storage unit 82 stores data used in operation of the server 80 and data obtained through operation of the server 80. For example, the storage unit 82 stores system programs, application programs, and embedded software. For example, the storage unit 82 stores data of the transformer 30 as illustrated in FIG. 6 and data used by the transformer 30.

The controller 83 includes at least one processor, at least one dedicated circuit, or a combination thereof. The processor can be a general-purpose processor such as a CPU or a GPU, or a dedicated processor specialized for particular processing. The dedicated circuit is, for example, an FPGA or an ASIC. The controller 83 executes processing relating to operation of the server 80 while controlling the various parts of the server 80. The controller 83 may perform the processing to be performed by the transformer 30 as illustrated in FIG. 6.

The controller 83 uses the communication unit 81 to receive sensor data from the electronic device 20 via the network 2. The controller 83 acquires estimated values of the ground reaction force applied to the user based on the sensor data by executing processing the same as or similar to the processing executed by the controller 27 of the electronic device 20 described above.

### (Other System Operations)

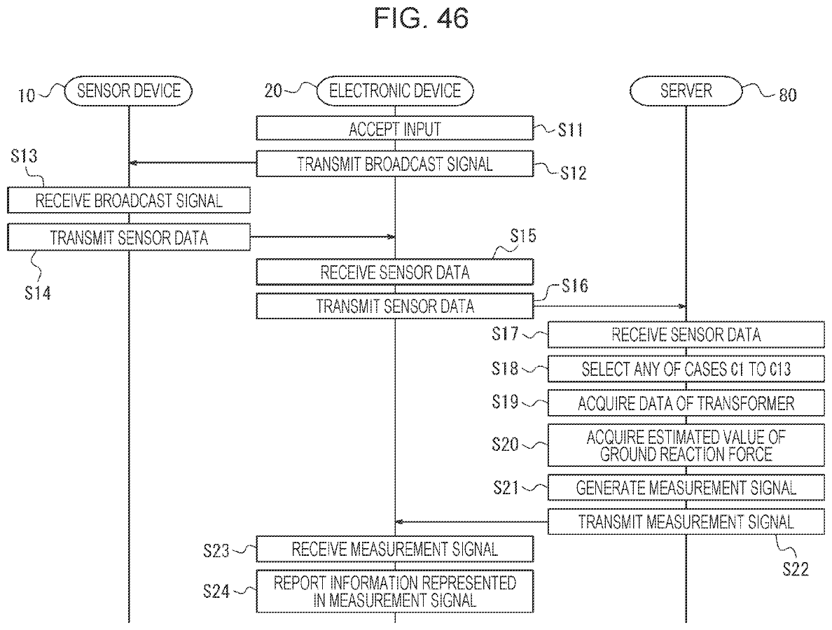

FIG. 46 is a sequence diagram illustrating operation of estimation processing performed by the information processing system illustrated in FIG. 45. This operation corresponds to an example of an information processing method according to this embodiment. When the electronic device 20 accepts an input instructing execution of the estimation processing for the ground reaction force, the information processing system 101 starts the estimation processing from the processing of Step S11.

In the electronic device 20, the controller 27 accepts an input instructing execution of the ground reaction force estimation processing via the input unit 22 (Step S11). The controller 27 transmits a signal instructing the start of data detection to the multiple sensor devices 10 as a broadcast signal via the communication unit 21 (Step S12).

In each sensor device 10, the controller 16 receives a signal instructing the start of data detection from the electronic device 20 via the communication unit 11 (Step S13). Upon receiving this signal, the controller 16 starts data detection. The controller 16 acquires data detected by the sensor unit 12 from the sensor unit 12 and transmits the acquired data as sensor data to the electronic device 20 via the communication unit 11 (Step S14).

In the electronic device 20, the controller 27 receives sensor data from the sensor device 10 via the communication unit 21 (Step S15). The controller 27 transmits the sensor data to the server 80 via the network 2 by using the communication unit 21 (Step S16).

In the server 80, the controller 83 receives the sensor data from the electronic device 20 via the network 2 by using the communication unit 81 (Step S17). The controller 83 selects any of the cases C1 to C13 in accordance with the type of sensor device 10 that transmitted the sensor data to the server 80 via the electronic device 20 (Step S18). The controller 83 acquires the data of the transformer 30 used in the cases C1 to C13 selected in the processing of Step S18 from the storage unit 82 (Step S19). The controller 83 inputs the sensor data for the one of the cases C1 to C13 selected in the processing of Step S18 to the transformer whose data was acquired in the processing of Step S19, and acquires estimated values of the ground reaction force from the transformer (Step S20). The controller 83 generates a measurement signal representing the estimated values of the ground reaction force (Step S21). The controller 83 transmits the generated measurement signal to the electronic device 20 via the network 2 by using the communication unit 81 (Step S22).

In the electronic device 20, the controller 27 receives the measurement signal from the server 80 via the network 2 by using the communication unit 21 (Step S23). The controller 27 causes the notification unit 23 to report the information represented by the measurement signal (Step S24). In the processing of Step S24, the controller 27 may transmit the measurement signal to the sensor device 10 by using the communication unit 21 and cause the sensor device 10 to report the information represented by the measurement signal.

After executing the processing of Step S24, the information processing system 101 terminates the estimation processing. After terminating the estimation processing, the information processing system 101 may perform the estimation processing again once the user has walked the set number of steps described above. In the estimation processing to be performed again, the information processing system 101 may start from the processing of Step S14. The information processing system 101 may repeat the evaluation processing each time the user takes the set number of steps until the electronic device 20 receives an input from the input unit 22 instructing termination of the estimation processing. As described above, upon receiving an input instructing termination of the estimation processing, the electronic device 20 may transmit a signal instructing termination of data detection to the multiple sensor devices 10 as a broadcast signal. As described above, upon receiving the signal instructing termination of data detection, each sensor device 10 may terminate the data detection.

In the processing of Step S20, the controller 83 of the server 80 may acquire information about the user's gait based on the estimated values of the ground reaction force, or may evaluate the user's gait. In this case, in the processing of Step S21, the controller 83 may generate a measurement signal representing at least any of the estimated values of the ground reaction force, evaluation of the user's gait, and information regarding the user's gait.

The information processing system 101 can achieve the same or similar effects to the information processing system 1.

The present disclosure has been described based on the drawings and examples, but note that a variety of variations and amendments may be easily made by one skilled in the art based on the present disclosure. Therefore, note that such variations and amendments are included within the scope of the present disclosure. For example, the functions and so forth included in each functional part can be rearranged in a logically consistent manner. Multiple functional parts and so forth may be combined into a single part or divided into multiple parts. Further, each embodiment according to the present disclosure described above does not need to be implemented exactly as described in the embodiment, and may be implemented with features having been combined or omitted as appropriate. A variety of variations and amendments to the content of the present disclosure can be made by one skilled in the art based on the present disclosure. Accordingly, such variations and amendments are included in the scope of the present disclosure. For example, in each embodiment, each functional part, each means, each step and so on can be added to other embodiments so long as there are no logical inconsistencies, or can be replaced with each functional part, each means, each step, and so on of other embodiments. In each embodiment, a plurality of each functional part, each means, each step, and so on can be combined into a single functional part, means, or step or divided into multiple functional parts, means, or steps. Each of the above-described embodiments of the present disclosure is not limited to faithful implementation of each of the described embodiments, and may be implemented by combining or omitting some of the features as appropriate.

For example, the communication unit 11 of the sensor device 10 may further include at least one communication module that can connect to the network 2 as illustrated in FIG. 45. The communication module is, for example, a communication module compatible with mobile communication standards such as LTE, 4G, or 5G. In this case, in the information processing system 101 as illustrated in FIG. 45, the controller 16 of the sensor device 10 may transmit the data detected by the sensor device 10 to the server 80 via the network 2 by using the communication unit 11.

For example, in the embodiment described above, the cases C5 to C8, C12, and C13 are described as including sensor data representing the user's wrist movement. However, in the cases C5 to C8, C12, and C13, instead of sensor data representing the movement of the user's wrist, sensor data representing the movement of a part of the user's forearm other than the wrist may be used.

For example, in the embodiment described above, the sensor device 10 is described as including the communication unit 11 as illustrated in FIG. 4 and FIG. 45. However, the sensor device 10 does not need to include the communication unit 11. In this case, sensor data detected by the sensor device 10 may be transferred, via a storage medium such as an SD (Secure Digital) memory card, to a device such as the electronic device 20 or the server 80 that will estimate the ground reaction force. SD memory cards are also called “SD cards”. The sensor device 10 may be configured to allow insertion of a storage medium such as an SD memory card.

For example, in the embodiment described above, the electronic device 20 or the server 80 is described as acquiring estimated values of the ground reaction force applied to the user by using sensor data detected by the sensor device 10 and a trained model. However, sensor data is not limited to sensor data detected by the sensor device 10 worn on a body part of the user, as long as the data represents the movement of a body part of the user. Sensor data may be detected using any method. As an example, the electronic device 20 or the server 80 may acquire estimated values of the ground reaction force applied to the user by using sensor data detected by motion capture, such as optical, image, or magnetic motion capture, and using a trained model.

For example, an embodiment in which a general-purpose computer is made to function as the electronic device 20 according to this embodiment is also possible. Specifically, a program describing processing content that realizes each function of the electronic device 20 according to this embodiment is stored in the memory of a general-purpose computer, and the program is read out and executed by a processor of the general-purpose computer. Therefore, the configuration according to this embodiment can also be realized as a program executable by a processor or a non-transitory computer-readable medium storing this program.

### REFERENCE SIGNS

1, 101 information processing system
2 network
10, 10A, 10B, 10C, 10D, 10D-1, 10D-2, 10E, 10E-1, 10E-2, 10F, 10F-1, 10F-2 sensor device
11 communication unit
12 sensor unit
13 notification unit
14 output unit
15 storage unit
16 controller
20 electronic device
21 communication unit
22 input unit
23 notification unit
24 output unit
25 vibration unit
26 storage unit
27 controller
30 transformer
40 encoder
41, 42, 44, 45, 46, 47 functional unit
43 layer
50 decoder
51, 52, 54, 55, 56, 57, 58, 59, 60, 61, 70 functional unit
53 layer
80 server
81 communication unit
82 storage unit
83 controller
C1, C2, C3, C4, C5, C6, C7, C8, C9, C10, C11, C12, C13 case
D10AG, D10AL, D10BL, D10CG, D10CL, D10DL-1, D10DL-2, D10EL-1, D10EL-2, D10FL-1, D10FL-2 sensor data

1. An information processing system comprising:
a controller configured to estimate a value of a ground reaction force applied to a user by using sensor data representing movement of a body part of the user and a trained model,
wherein the trained model is trained and outputs the value of the ground reaction force when input with the sensor data.

2. The information processing system according to claim 1,
wherein the controller is configured to acquire the sensor data from at least one sensor device worn on the body part of the user.

3. The information processing system according to claim 1,
wherein the trained model is trained and outputs the value of the ground reaction force when input with the sensor data, the sensor data representing movement of a head of the user.

4. The information processing system according to claim 1,
wherein the trained model is trained and outputs the value of the ground reaction force when input with the sensor data, the sensor data including sensor data representing movement of a head of the user and sensor data representing movement of either one of two ankles of the user.

5. The information processing system according to claim 1,
wherein the trained model is trained and outputs the value of the ground reaction force when input with the sensor data, the sensor data including sensor data representing movement of a head of the user and sensor data representing movement of either one of two feet of the user.

6. The information processing system according to claim 1,
wherein the trained model is trained and outputs the value of the ground reaction force when input with the sensor data, the sensor data including sensor data representing movement of a head of the user and sensor data representing movement of either one of two thighs of the user.

7. The information processing system according to claim 1,
wherein the trained model is trained and outputs the value of the ground reaction force when input with the sensor data, the sensor data including sensor data representing movement of a head of the user and sensor data representing movement of either one of two forearm of the user.

8. The information processing system according to claim 1,
wherein the trained model is trained and outputs the value of the ground reaction force when input with the sensor data, the sensor data including sensor data representing movement of a head of the user, sensor data representing movement of either one of two forearms of the user, and sensor data representing movement of either one of two ankles of the user.

9. The information processing system according to claim 1,
wherein the trained model is trained and outputs the value of the ground reaction force when input with the sensor data, the sensor data including sensor data representing movement of a head of the user, sensor data representing movement of either one of two forearms of the user, and sensor data representing movement of either one of two feet of the user.

10. The information processing system according to claim 1,
wherein the trained model is configured to estimate a value of a normalized ground reaction force obtained by normalizing a ground reaction force, and
the controller is configured to calculate a calculated value of the ground reaction force by multiplying the value of the normalized ground reaction force by a weight of the user.

11. The information processing system according to claim 1,
wherein the trained model is a transformer.

12. The information processing system according to claim 1,
wherein the controller is configured to evaluate a gait of the user based on the value of the ground reaction force.

13. The information processing system according to claim 1,
wherein the controller is configured to acquire information about a gait of the user based on the value of the ground reaction force.

14. The information processing system according to claim 1, further comprising:
a communication unit,
wherein the controller is configured to generate a measurement signal representing at least any of the value of the ground reaction force, an evaluation of a gait of the user, and information about the gait of the user, and to transmit the measurement signal to an external device by using the communication unit.

15. The information processing system according to claim 14,
wherein the external device is an earphone.

16. The information processing system according to claim 1,
wherein the trained model is generated using, as training data, a data set including data on ground reaction forces of multiple subjects detected by a floor reaction force meter and data representing movements of subjects detected by a motion capture system.

17. An electronic system comprising:
a notification unit configured to report the value of the ground reaction force acquired by the information processing device according to claim 1.

18. (canceled)

19. An information processing method comprising:
estimating a value of a ground reaction force applied to a user by using sensor data representing movement of a body part of the user and a trained model,
wherein the trained model is trained and outputs the value of the ground reaction force when input with the sensor data.

20. A program configured to cause a computer to execute estimating a value of a ground reaction force applied to a user by using sensor data representing movement of a body part of the user and a trained model,
wherein the trained model is trained and outputs the value of the ground reaction force when input with the sensor data.
